SEQUENCE LISTING (14 sequences)

SEQ ID NO: 1 - DNA, 750 bp, Artificial Sequence
  Description of Artificial Sequence DR2-Fos fusion
gtatctctcgagaaaagagagatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacttggaaatcatgacaaagcgctccaactatactccgatcaccaatgtacctccagaggtaactgtgctcacgaacagccctgtggaactgagagagcccaacgtcctcatctgtttcatagacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactgggagtttgatgctccaagccctctcccagagactacagaggtcgacggaggtggcggcggtttaactgatacactccaagcggagacagatcaacttgaagacgagaagtctgcgttgcagaccgagattgccaatctactgaaagagaaggaaaaactggagttcatcctggccgcccattgagaattctatgac

SEQ ID NO: 2 - protein, 245 aa, Artificial Sequence
  Description of Artificial Sequence DR2-Fos fusion
VSLEKREIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTEVDGGGGGLTDTLQAETDQLEDEKSALQTEIANLLKEKEKLEFILAAH

SEQ ID NO: 3 - DNA, 771 bp, Artificial Sequence
  Description of Artificial Sequence DR2-Jun fusion
gtatctctcgagaaaagagagggggacacccgaccacgtttcctgtggcagcctaagagggagtgtcatttcttcaatgggacggagcgggtgcggttcctggacagatacttctataaccaggaggagtccgtgcgcttcgacagcgacgtgggggagttccgggcggtgacggagctggggcggcctgacgctgagtactggaacagccagaaggacatcctggagcaggcgcgggccgcggtggacacctactgcagacacaactacggggttgtggagagcttcacagtgcagcggcgagtccaacctaaggtgactgtatatccttcaaagacccagcccctgcagcaccacaacctcctggtctgctctgtgagtggtttctatccaggcagcattgaagtcaggtggttcctgaacggccaggaagagaaggctgggatggtgtccacaggcctgatccagaatggagactggaccttccagaccctggtgatgctggaaacagttcctcgaagtggagaggtttacacctgccaagtggagcacccaagcgtgacaagccctctcacagtggaatggagagcacggtctgaatctgcacagagcaaggtcgacggaggtggcggcggtcgcatcgcccggctcgaggaaaaagtgaaaaccttgaaagctcagaactcggagctcgcgtccacggccaacatgctcagggaacaggtggcacagcttaaacagaaagtcatgaaccattgagaattctatgac

SEQ ID NO: 4 - protein, 252 aa, Artificial Sequence
  Description of Artificial Sequence DR2-Jun fusion
VSLEKREGDTRPRFLWQPKRECHFFNGTERVRFLDRYFYNQEESVRFDSDVGEFRAVTELGRPDAEYWNSQKDILEQARAAVDTYCRHNYGVVESFTVQRRVQPKVTVYPSKTQPLQHHNLLVCSVSGFYPGSIEVRWFLNGQEEKAGMVSTGLIQNGDWTFQTLVMLETVPRSGEVYTCQVEHPSVTSPLTVEWRARSESAQSKVDGGGGGRIARLEEKVKTLKAQNSELASTANMLREQVAQLKQKVMNH

SEQ ID NO: 5 - DNA, 42 bp, Artificial Sequence
  Description of Artificial Sequence Synthetic PCR primer
gtatctctcgagaaaagagagatcaaagaagaacatgtgatc

SEQ ID NO: 6 - DNA, 39 bp, Artificial Sequence
  Description of Artificial Sequence Synthetic PCR primer
gtcatagaattctcaatgggcggccaggatgaactccag

SEQ ID NO: 7 - DNA, 42 bp, Artificial Sequence
  Description of Artificial Sequence Synthetic PCR primer
gtatctctcgagaaaagagagggggacacccgaccacgtttc

SEQ ID NO: 8 - DNA, 39 bp, Artificial Sequence
  Description of Artificial Sequence Synthetic PCR primer
gtcatagaattctcaatggttcatgactttctgtttaag

SEQ ID NO: 9 - protein, 14 aa, Artificial Sequence
  Description of Artificial Sequence Synthetic, biotin ligase recognition sequence
LGGIFEAMKMELRD

SEQ ID NO: 10 - protein, 16 aa, Artificial Sequence
  Description of Artificial Sequence Synthetic, linker sequence
SGGGSLVPRGSGGGGS

SEQ ID NO: 11 - DNA, 1446 bp, Artificial Sequence
  Description of Artificial Sequence DR2-IgG fusion
ctcgagaaaagagagatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacttggaaatcatgacaaagcgctccaactatactccgatcaccaatgtacctccagaggtaactgtgctcacgaacagccctgtggaactgagagagcccaacgtcctcatctgtttcatagacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactgggagtttgatgctccaagccctctcccagagactacagaggtcgacggaggtggcggcggtttaactgatacactccaagcggagacagatcaacttgaagacgagaagtctgcgttgcagaccgagattgccaatctactgaaagagaaggaaaaactggagttcatcctggccgcccatgcagcatctgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatgagaattc

SEQ ID NO: 12 - protein, 479 aa, Artificial Sequence
  Description of Artificial Sequence DR2-IgG fusion
LEKREIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTEVDGGGGGLTDTLQAETDQLEDEKSALQTEIANLLKEKEKLEFILAAHAASEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 13 - DNA, 1851 bp, Artificial Sequence
  Description of Artificial Sequence DR2-IgM fusion
atggccataagtggagtccctgtgctaggatttttcatcatagctgtgctgatgagcgctcaggaatcatgggctatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacttggaaatcatgacaaagcgctccaactatactccgatcaccaatgtacctccagaggtaactgtgctcacgaacagccctgtggaactgagagagcccaacgtcctcatctgtttcatagacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactgggagtttgatgctccaagccctctcccagagactacagaggtcgacggaggtggcggcggtttaactgatacactccaagcggagacagatcaacttgaagacgagaagtctgcgttgcagaccgagattgccaatctactgaaagagaaggaaaaactggagttcatcctggccgcccacgtcgcagaaatgaaccccaatgtaaatgtgttcgtcccaccacgggatggcttctctggccctgcaccacgcaagtctaaactcatctgcgaggccacgaacttcactccaaaaccgatcacagtatcctggctaaaggatgggaagctcgtggaatctggcttcaccacagatccggtgaccatcgagaacaaaggatccacaccccaaacctacaaggtcataagcacacttaccatctctgaaatcgactggctgaacctgaatgtgtacacctgccgtgtggatcacaggggtctcaccttcttgaagaacgtgtcctccacatgtgctgccagtccctccacagatatccttaattttactattcctccttcctttgccgacatcttccttagcaagtccgctaacctgacctgtctggtctcaaacctggcaacctatgaaaccctgagtatctcctgggcttctcaaagtggtgaaccactggaaaccaaaattaaaatcatggaaagccatcccaatggcaccttcagtgctaagggtgtggctagtgtttgtgtggaagactggaataacaggaaggaatttgtgtgtactgtgactcacagggatctgccttcaccacagaagaaattcatctcaaaacccaatgaggtgcacaaacatccacctgctgtgtacctgctgccaccagctcgtgaacaactgaacctgagggagtcagccacagtcacctgcctggtgaagggcttctctcctgcagacatctctgtgcaatggaagcagaggggccagctcttaccccaggagaagtatgtgaccagtgccccgatgccagagcctggggccccaggcttctactttacccacagcatcctgactgtgacagaggaggaatggaactccggagagacctatacctgtgttgtaggccacgaggccctgccacacctggtgaccgagaggaccgtggacaagtccactggtaaacccacactgtacaatgtctccctgatcatgtctgacacaggcggcacctgctattgaagatctgtcgac

SEQ ID NO: 14 - protein, 612 aa, Artificial Sequence
  Description of Artificial Sequence DR2-IgM fusion
MAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTEVDGGGGGLTDTLQAETDQLEDEKSALQTEIANLLKEKEKLEFILAAHVAEMNPNVNVFVPPRDGFSGPAPRKSKLICEATNFTPKPITVSWLKDGKLVESGFTTDPVTIENKGSTPQTYKVISTLTISEIDWLNLNVYTCRVDHRGLTFLKNVSSTCAASPSTDILNFTIPPSFADIFLSKSANLTCLVSNLATYETLSISWASQSGEPLETKIKIMESHPNGTFSAKGVASVCVEDWNNRKEFVCTVTHRDLPSPQKKFISKPNEVHKHPPAVYLLPPAREQLNLRESATVTCLVKGFSPADISVQWKQRGQLLPQEKYVTSAPMPEPGAPGFYFTHSILTVTEEEWNSGETYTCVVGHEALPHLVTERTVDKSTGKPTLYNVSLIMSDTGGTCY

(19) United States
(12) Patent Application Publication
(10) Pub. No.: US 2005/0003431 A1
(43) Pub. Date: Jan. 6, 2005

(54) Monovalent, multivalent, and multimeric MHC binding domain fusion proteins and conjugates, and uses therefor

(76) Inventors: Kai W. Wucherpfennig, Brookline, MA (US); Jack L. Strominger, Lexington, MA (US)

Correspondence Address: ROPES & GRAY LLP, ONE INTERNATIONAL PLACE, BOSTON, MA, 02110-2624, US

(21) Appl. No.: 10/895,543

(22) Filed: Jul. 21, 2004

Related U.S. Application Data
(62) Division of application No. 09/248,964, filed on Feb. 12, 1999.
(63) Continuation-in-part of application No. PCT/US97/14503, filed on Aug. 15, 1997.
(60) Provisional application No. 60/075,351, filed on Feb. 19, 1998.
(60) Provisional application No. 60/024,077, filed on Aug. 16, 1996.

(51) Int. Cl. C12Q 1/68; C07H 21/04; C12P 21/04; C07K 14/74

(52) U.S. Cl. 435/6; 435/69.7; 435/320.1; 435/325; 530/350; 536/23.5

(57) ABSTRACT

The present invention is directed to the field of immunology. In particular, the present invention is directed to the design, production, and use of monovalent, multivalent and multimeric Major Histocompatibility Complex binding domain fusion proteins and conjugates.

### RELATED APPLICATIONS

This application claims priority to U.S. Provisional Appln. Ser. No. 60/075,351, filed Feb. 19, 1998, and is a continuation-in-part of International Application PCT/US97/14503, with international filing date Aug. 15, 1997, which claimed priority to U.S. Provisional Appln. Ser. No. 60/024,077, filed Aug. 16, 1996.

### GOVERNMENT SUPPORT

The present invention was made, in part, with support from the National Institutes of Health Grant No. CA47554. The government may have certain rights in the invention.

### FIELD OF THE INVENTION

The present invention is directed to the field of immunology. In particular, the present invention is directed to the design, production, and use of Major Histocompatibility Complex binding domain fusion proteins and conjugates.

### BACKGROUND OF THE INVENTION

MHC molecules are highly polymorphic dimeric proteins which determine the specificity of T cell mediated immune responses by binding peptides from foreign antigens in an intracellular processing compartment, and by presenting these peptides on the surface of antigen presenting cells, where they may be recognized by specialized T cell receptors (TCRs) (reviewed in Strominger and Wiley, 1995). For example, the MHC Class II DR β chain gene, with 137 known DRB1 alleles (Marsh and Bodmer, 1995), is the most polymorphic human gene that has been identified. Not surprisingly, the polymorphic residues of these proteins are clustered in peptide binding domains which define the large repertoire of peptides that may be presented to T cells (Bjorkman et al., 1987; Stem et al., 1994). Although T cells should not normally react to self peptides presented in syngeneic MHC molecules, some alleles of the MHC genes are believed to confer susceptibility to autoimmune diseases through the presentation of pathogenic self-peptides. Thus, for example, the MHC Class II BLA-DR2 subtypes confer an increased risk for multiple sclerosis (MS), while subtypes of HLA-DR4 confer susceptibility to rheumatoid arthritis (reviewed in Todd et al., 1988; Wucherpfennig and Strominger, 1995b).

The production of soluble, “empty” MHC Class II molecules (i.e., molecules which do not have peptides bound within the MHC Class II peptide binding domains) would be highly useful in producing homogeneous preparations of MHC/peptide complexes “loaded” with a single variety of peptide. Such soluble, MHC/peptide complexes have several important investigational and therapeutic uses. For example, soluble MHC Class II molecules are required for crystallographic studies of single MHC/peptide complexes, and for studying the biochemical interaction of particular MHC/peptide complexes with their cognate TCRs. Structural characterization of the MHC/peptide/TCR recognition unit will provide important insights into the mechanisms by which MHC molecules confer susceptibility to autoimmunity. In addition, soluble MHC/peptide complexes are useful for the treatment of autoimmune diseases. For example, studies in the murine experimental autoimmune encephalomyelitis (EAE) model have demonstrated that an autoimmune disease can be treated by the administration of soluble MHC/peptide complexes loaded with the autoantigenic peptide (Sharma et al., 1991). Such complexes are expected to be useful in the treatment of several human autoimmune diseases, including multiple sclerosis (MS) and rheumatoid arthritis (RA).

A number of approaches have been followed to obtain purified, soluble, empty MHC Class II molecules. For example, MHC Class II molecules can be purified from mammalian cells by affinity chromatography following detergent solubilization of B cell membranes (Gorga et al., 1987). MHC molecules purified from B cell lines, however, have already passed through the intracellular MHC Class II peptide loading compartment and, therefore, are already loaded with a diverse set of peptides (Chicz et al., 1992). Furthermore, removal of these peptides from B cell derived MHC complexes (e.g., by low pH treatment) is very difficult and typically results in MHC protein denaturation. In another approach, soluble, truncated HLA-DR1 and HLA-DR4 molecules have been expressed in the baculovirus/insect cell system using cDNA constructs for the DRα and DRβ extracellular domains without the hydrophobic transmembrane domains (Stern and Wiley, 1992). These molecules were assembled and secreted but had a tendency to aggregate unless they were loaded with a high affinity peptide. Moreover, this approach has not been successful with HLA-DR2 molecules. For example, the product of the DRA, DRB5*0101 genes showed a strong tendency to aggregate even when high affinity peptides were added (Vranovsky and Strominger, unpublished observations). In addition, when this approach was attempted with the DR2 molecules formed by the DRA and DRB1*1501 gene products, the DRα and DRβ chains failed to assemble (Wucherpfennig, unpublished observations). In yet another approach, Wettstein et al. (1991) expressed a murine Class II heterodimer (E^{k}) as a glycan-phosphatidylinositol linked chimera which could be cleaved from CHO cells by phospholipase C to yield a soluble form, but this form required 100-fold higher concentrations of peptide to yield two- to four-fold lower levels of T cell stimulation. The expression of soluble mouse I-A molecules (1-Au and I-A^{g7}, which confer susceptibility to EAE and diabetes, respectively) has also been difficult. When the extracellular domains of these MHC molecules were fused with a glycan-phosphatidyl inositol anchor and then cleaved from the surface of transfected cells, irreversible aggregation occurred even if the cells had been incubated with I-A binding peptides prior to cleavage (L. Fugger and H. McDevitt, personal communication). All of these observations with truncated MHC molecules suggest that, for some but not all of these proteins, the α-helical transmembrane regions of the MHC Class II α and β chains are essential to the normal assembly of the αβ heterodimer (Cosson and Bonifacino, 1992).

It has been suggested that “dimerization domains” of known, stable dimeric proteins may be genetically engineered into fusion proteins to promote the formation of stable dimeric fusion proteins. For example, synthetic peptides of the isolated Fos and Jun leucine zipper dimerization domains, with added N-terminal cysteine residues and (Gly)₂ linkers, were shown to assemble as soluble heterodimers with interchain disulfide bridges (O'Shea et al., 1989). Fusion proteins including artificial leucine zipper dimerization domains were also employed to express αβ heterodimers of the TCR extracellular domains with interchain disulfide bridges (Chang et al., 1994). Although these TCR chimeras were bound by antibodies to native TCRs, they were not shown to retain MHC/peptide complex specificity. In another approach, Gregiore et al. (1991) produced soluble αβ heterodimers of TCR extracellular domains by co-expressing proteins in which the variable and constant (first exon only) domains of α and β TCR chains were each fused to the same constant domain of an immunoglobulin K light chain. Again, although the fusion heterodimers were recognized by antibodies to the native TCR, these authors were unable to measure direct binding of the fusion heterodimer to its cognate MHC antigen, and found that the fusion heterodimer failed to reproducibly inhibit T cells bearing the native TCR from recognizing cells bearing the cognate MHC antigen. Finally, Weber et al. (1992), using a similar approach, failed to detect direct MHC binding of a TCR fusion heterodimer but inferred low affinity binding from binding competition experiments.

### SUMMARY OF THE INVENTION

The present invention is directed to monovalent and multivalent fusion proteins, and multimeric protein conjugates, comprising human Major Histocompatibility Complex binding domains, with or without bound MHC binding peptides, which are useful in diagnostic and therapeutic methods, as well as laboratory assays.

In one aspect, the present invention provides MHC binding domain fusion proteins of MHC Class II α and β chain proteins in which substantially all of the C-terminal transmembrane and cytoplasmic domains have been replaced by dimerization domains and, optionally, interposing linker sequences.

Thus, a Class II MHC binding domain fusion protein is provided comprising a fusion of, toward the N-terminus, at least an MHC. Class II binding domain of an MHC Class II α chain and, toward the C-terminus, a dimerization domain. In preferred embodiments, the MHC Class II binding domain comprises an extracellular domain of an MHC Class II α chain, preferably at least residues 5-180 of an MHC Class III α chain, more preferably residues 5-190, and most preferably residues 5-200. The MHC Class II α chains from which the fusion proteins of the invention may be derived include HLA-DR1, HLA-DR2, HLA-DR4, HLA-DQ1, HLA-DQ2 and HLA-DQ8 cc chains, and particularly α chains encoded by DRA*0101, DRA*0102, DQA1*0301 or DQA1*0501 alleles.

Similarly, a Class II MHC binding domain fusion protein is provided comprising a fusion of, toward the N-terminus, at least an MHC Class II binding domain of an MHC Class II β chain and, toward the C-terminus, a dimerization domain. In preferred embodiments, the MHC Class II binding domain comprises an extracellular domain of an MHC Class II β chain, preferably at least residues 5-185 of an MHC Class II H chain, more preferably residues 5-195, and most preferably residues 5-205. The MHC Class II p chains from which the fusion proteins of the invention may be derived include LA-DR1, LA-DR2, HLA-DR4, ILA-DQ1, BLA-DQ2 and HLA-DQ8 p chains, and particularly β chains encoded by DRB1*01, DRB1*15, DRB1*16, DRB5*01, DQB1*03 and DQB1*02 alleles.

In some preferred embodiments, the dimerization domains of the Class II MHC binding domain fusion proteins comprise coiled-coil dimerization domains, such as leucine zipper domains. Preferably, the leucine zipper domains include at least four leucine heptads. In one preferred embodiment, the leucine zipper domain is a Fos or Jun leucine zipper domain.

In other embodiments, the dimerization domain is an immunoglobulin Fab constant domain, such as an immunoglobulin heavy chain C_{H}1 constant region or an immunoglobulin light chain constant region.

In each of the foregoing embodiments, a flexible molecular linker optionally may be interposed between, and covalently join, the MHC Class II binding domain and the dimerization domain. Preferably, the flexible molecular linker comprises a peptide sequence of 1-15 amino acid residues, more preferably 5-7 amino acid residues. In addition, when polypeptide linkers are employed, it is preferred that a majority of the amino acid residues in the linker are alanine, glycine, serine, leucine, isoleucine, or valine residues.

In addition, in each of the foregoing embodiments, an MHC Class II binding peptide optionally may be covalently joined to the N-terminus of the MHC Class II α or β chain binding domain, such that the binding peptide is capable of selectively binding to an MHC Class II binding domain formed by the α or β chain and another (β or α, respectively) MHC Class II chain. Thus, the MHC binding peptide and the MHC Class II binding domain form an MHC/peptide complex. Preferably, the MHC binding peptide is joined to the N-terminus of the β chain. Essentially any MHC binding peptides may be joined to the N-termini of MHC Class II chains with which they selectively bind in nature. In particularly preferred embodiments with medical importance to multiple sclerosis, however, the MUC Class II binding domain is an BLA-DR2 binding domain and the binding peptide is selected from residues 85-99, 84-102 and 148-162 of human myelin basic protein. Similarly, in particularly preferred embodiments with medical importance to pemphigus vulgaris, the MHC Class II binding domain is an HLA-DR4 or HLA-DQ1 binding domain and said binding peptide is selected from residues 78-93, 97-111, 190-204, 206-220, 251-265, 512-526 and 762-786 of the human desmoglein 3 protein.

In each of the foregoing embodiments employing a covalently bound MHC binding peptide in the fusion protein, a flexible molecular linker optionally may be interposed between, and covalently join, the MHC Class II chain and the MHC binding peptide. Preferably, the linker is a polypeptide sequence of 10-20 amino acid residues, more preferably 12-18 amino acid residues. When a polypeptide linker is employed, it is preferred that a majority of the amino acid residues are alanine, glycine, serine, leucine, isoleucine, and valine residues.

In another aspect, the present invention provides Class II MHC binding domain fusion proteins comprising a heterodimer of a first polypeptide chain and a second polypeptide chain, in which the first polypeptide chain comprises a fusion of, toward the N-terminus, at least an MHC binding domain of an MHC Class II α chain and, toward the C-terminus, a first dimerization domain, and the second polypeptide chain comprises a fusion of, toward the N-terminus, at least an MHC binding domain of an MHC Class II β chain and, toward the C-terminus, a second dimerization domain. In these embodiments, the first dimerization domain and the second dimerization domain associate in solution at physiological conditions to form a heterodimer capable of selectively binding an MHC binding peptide. The dimerization domains, as described above, may be coiled-coil dimerization domains and, preferably, leucine zipper domains. Flexible molecular linkers, as described above, may be interposed between and covalently join the MHC chains and dimerization domains, and MHC binding peptides may be covalently joined to one of the MHC chains.

In another aspect, a Class II MHC binding domain fusion protein is provided comprising a heterodimer of a first polypeptide chain and a second polypeptide chain, in which the first polypeptide chain comprises a fusion of, toward the N-terminus, at least an MHC binding domain of an MHC Class II α chain and, toward the C-terminus, an immunoglobulin heavy chain C_{H}1 constant region, and the second polypeptide chain comprises a fusion of, toward the N-terminus, at least an MHC binding domain of an MHC Class II β chain and, toward the C-terminus, an immunoglobulin light chain constant region. In these embodiments, the immunoglobulin heavy chain C_{H}1 constant region and the immunoglobulin light chain constant region dimerize in solution at physiological conditions to form a heterodimer capable of selectively binding an MHC binding peptide. Alternatively, a Class II MHC binding domain fusion protein is provided comprising a heterodimer of a first polypeptide chain and a second polypeptide chain, in which the first polypeptide chain comprises a fusion of, toward the N-terminus, at least an extracellular domain of an MHC Class II α chain and, toward the C-terminus, an immunoglobulin light chain constant region, and the second polypeptide chain comprises a fusion of, toward the N-terminus, at least an extracellular domain of an MHC Class II β chain and, toward the C-terminus, an immunoglobulin heavy chain C_{H}1 constant region. In these embodiments, as above, the immunoglobulin heavy chain C_{H}1 constant region and the immunoglobulin light chain constant region dimerize in solution at physiological conditions to form a heterodimer capable of selectively binding an MHC binding peptide. In each of these embodiments, the Class II MHC fusion protein may further comprise an immunoglobulin Fc region covalently joined to the immunoglobulin heavy chain CHI constant region. Such Fc regions may be IgE or IgM Fc regions, and a flexible molecular linker may optionally be interposed between, and covalently join, the immunoglobulin heavy chain C_{H}1 constant region and immunoglobulin Fc region. Alternatively, the Fc regions may be IgA, IgD or IgG Fc regions. As before, a flexible molecular linker may be optionally interposed between, and covalently join, the immunoglobulin heavy chain C_{H}1 constant region and immunoglobulin Fc region and, in these embodiments, may be immunoglobulin hinge regions.

In particularly preferred embodiments, a multivalent Class II MHC binding domain fusion protein is provided comprising two Class II MHC binding domain fusion proteins as described above, in which the Fc regions are covalently joined by at least one disulfide bond. Most preferably, a multivalent Class II MHC binding domain fusion protein is provided comprising five pairs of Class II MHC binding domain fusion proteins in which the Fc regions are IgM regions, each pair is covalently joined by at least one disulfide bond between Fc regions of each pair, and the five pairs are covalently joined by disulfide bridges to form a ring structure such that each adjacent pair in the ring is joined by at least one disulfide bond.

In each of the foregoing embodiments, the Class II MHC binding domain fusion proteins may further comprise an N-terminal secretory signal sequence covalently joined to the N-terminus of the fusion protein. In preferred embodiments, the secretory signal sequence comprises a yeast α-mating factor secretion signal or a human MHC Class II protein secretion signal.

In another aspect, the present invention provides for multimeric MHC binding domain conjugates comprising a carrier conjugated to a multiplicity of MHC binding domains, with or without peptide bound thereto.

In some preferred embodiments, the multimeric MHC binding domain conjugates comprise about 5 to about 500 MHC binding domains per carrier, preferably about 10 to about 200 MHC binding domains per carrier, and most preferably about 20 to about 100 MHC binding domains per carrier.

In some preferred embodiments, the multimeric MHC binding domain conjugates are characterized by the presence of MHC binding domains at an average density of about 4×10⁻³ to 20 MHC binding domains/nm² on the surface of the carrier, preferably about 4×10-2 to 20 MHC binding domains/nm², and most preferably about 0.4 to 20 MHC binding domains/nm² on said surface.

In some preferred embodiments, the multimeric MHC binding domain conjugates comprise a carrier having a maximum diameter of about 5 to about 1000 nm, preferably about 5 to about 500 nm, and most preferably about 5 to about 100 nm. In some embodiments, the multimeric MHC binding domain conjugates define a minimal surface area of less than approximately 3.1×10⁶ nm², preferably less than 7.9×10⁵ nm², and more preferably less than 3.1×10⁴ nm². In most preferred embodiments, MHC binding domain conjugates define minimal surface areas of approxiamtely 78.5 to 5.0×10³ m². In some embodiments, the multimeric MHC binding domain conjugates define a minimal volume of less than approximately 5.2×10⁸ nm³, preerably less than 6.5×10⁷ nm³, and more preferably less than 5.2×10⁵ nm³. In most preferred embodiments, MC binding domain conjugates define minimal volumes of 65.4 to 3.4×10⁴ nm³.

In some preferred embodiments, the multimeric MHC binding domain conjugates comprise a carrier weighing about 100 kDa to about 10,000 kDa, preferably about 100 kDa to about 5,000 kDa, more preferably about 100 kDa to about 1,000 kDa, and most preferably about 100 kDa to about 500 kDa.

In some preferred embodiments, the multimeric MHC binding domain conjugates weigh about 400 kDa to about 10,000 kDa, preferably about 400 kDa to about 5,000 kDa, more preferably about 400 kDa to about 1,000 kDa, and most preferably about 400 kDa to about 500 kDa.

In some preferred embodiments, the multimeric MHC binding domain conjugate is particulate, the carrier is biodegradable, the carrier is non-immunogenic, the carrier has a net neutral or negative charge, and/or the carrier is fluorescently labeled. The carrier may be covalently or non-covalently bound to the MHC binding domains.

In some embodiments, the multimeric MHC binding domain conjugate comprises a carrier which is a substantially spherical bead or a porous bead. In preferred embodiments in which the carrier is a bead, the bead preferably comprises a material selected from the group consisting of glass, silica, polyesters of hydroxy carboxylic acids, polyanhydrides of dicarboxylic acids, or copolymers of hydroxy carboxylic acids and dicarboxylic acids.

In some embodiments, the multimeric MHC binding domain conjugate comprises a carrier which is a branched polymer, such as a dendrimer. In preferred embodiments when the carrier is a dendrimer, the dendrimer comprises a material selected from the group consisting of a polyamidoamine, a polyamidoalcohol, a polyalkyleneimine, a polyalkylene, a polyether, a polythioether, a polyphosphonium, a polysiloxane, a polyamide, and a polyaryl polymer.

In some embodiments, the multimeric MHC binding domain conjugate comprises a carrier which is a liposome. In these embodiments, the liposome preferably comprises a material selected from the group consisting of phosphatidyl choline, phosphatidyl serine, phosphatidyl inositol, phosphatidyl glycerol, phosphatidyl ethanolamine, phosphatidic acid, dicetyl phosphate, monosialoganglioside, polyethylene glycol, stearyl amine, ovolecithin and cholesterol.

In each of the foregoing embodiments, the multimeric MHC binding domain conjugates may further comprise a multiplicity of MHC binding peptides bound to the MHC binding domains, either covalently or non-covalently.

In each of the foregoing embodiments of multimeric MHC binding domain conjugates, each MHC binding domain preferably comprises a heterodimer of at least the peptide binding domain of an MHC Class I α chain and an MHC Class β chain, or a heterodimer of at least the peptide binding domain of an MHC Class II α chain and an MHC Class II β chain. Further, in each of these embodiments, the MHC binding domains may comprise a part of a monovalent or multivalent MHC binding domain fusion protein of the invention.

In another aspect, the present invention provides a method for detecting T cells having a defined MHC/peptide-complex specificity comprising providing a monovalent, multivalent or multimeric MHC binding domain fusion protein or conjugate, as described above and comprising the defined MHC/peptide complex, contacting a population of T cells with the fusion protein or conjugate, and detecting the presence or absence of binding of the fusion protein or conjugate and T cells in the population. Also provided is a method further comprising isolating T cells reactive with the defined MHC/peptide complex from the population of T cells by, for example, means of fluorescence activated cell sorting.

In another aspect, the present invention provides a method of conferring to a subject adoptive immunity to a defined MHC/peptide complex comprising providing a monovalent, multivalent or multimeric MHC binding domain fusion protein or conjugate, as described above and comprising the defined MHC/peptide complex, contacting a population of T cells with the fusion protein or conjugate, isolating T cells reactive with the defined MHC/peptide complex from the population of T cells, and administering the isolated T cells to the subject to provide adoptive immunity.

In another aspect, the present invention provides a method for stimulating or activating T cells reactive to a defined MHC/peptide complex comprising providing a monovalent, multivalent or multimeric MHC binding domain fusion protein or conjugate, as described above and comprising the defined MHC/peptide-complex, and contacting a population of T cells with an immunogenic amount of the fusion protein or conjugate. In preferred embodiments, the fusion protein or conjugate is contacted with the population of T cells in vivo in a human subject, and the MHC fusion protein or conjugate comprises an MHC binding domain which is syngeneic to the subject.

In another aspect, the present invention provides a method for selectively killing T cells reactive to a defined MHC/peptide complex comprising providing a monovalent, multivalent or multimeric MHC binding domain fusion protein or conjugate, as described above and comprising the defined MHC/peptide-complex, and contacting a population of T cells with the fusion protein or conjugate, in which the fusion protein or conjugate comprises a domain of an immunoglobulin effective to activate the complement system and cause the complement system to kill the T cells.

In another aspect, the present invention provides a method for selectively killing T cells reactive to a defined MHC/peptide complex comprising providing a monovalent, multivalent or multimeric MHC binding domain fusion protein or conjugate, as described above and comprising the defined MHC/peptide-complex, and contacting a population of T cells with the fusion protein or conjugate, in which the fusion protein or conjugate comprises a cytotoxic substance associated with the protein or conjugate and capable of killing T cells to which the fusion protein or conjugate selectively binds.

In another aspect, the present invention provides a method for tolerizing a human subject to a defined MHC/peptide complex comprising providing a monovalent, multivalent or multimeric MHC binding domain fusion protein or conjugate, as described above and comprising the defined MHC/peptide-complex, and administering to the subject an amount of the fusion protein or conjugate effective to induce tolerization to said MHC/peptide complex. In certain preferred embodiments, the MHC fusion protein or conjugate comprises an MHC binding domain which is syngeneic to the subject. In other preferred embodiments, however, the MHC fusion protein or conjugate comprises an MHC binding domain which is allogeneic to the subject.

In another aspect the present invention provides nucleic acid sequences encoding the above-described MHC binding domain fusion proteins.

### BRIEF DESCRIPTION OF THE DRAWINGS

FIG. 1. This figure is a schematic representation of one embodiment of a monovalent MHC binding domain fusion protein of the invention. Here, an extracellular or peptide binding domain of an MHC Class II ax chain 10 is joined to a first dimerization domain 30, an extracellular or peptide binding domain of an MHC Class II D chain 20 is joined to a second dimerization domain 40, and these two fusion constructs form a heterodimeric molecule which binds an MHC binding peptide 110 in the cleft formed by the MHC Class II binding domains of the α and β chains, 10 and 20. Optionally, flexible molecular linkers, not shown, are interposed between the MHC domains (10, 20) and the dimerization domains (30, 40).

FIG. 2. This figure is a schematic representation of one embodiment of a divalent MHC binding domain fusion protein construct of the invention. Here, an extracellular or peptide binding domain of an MC Class II α chain 10 is joined to either a first coiled-coil or dimerization domain or an immunoglobulin heavy chain C_{H}1 constant region 30, and an extracellular or peptide binding domain of an MHC Class II β chain 20 is joined to a second coiled-coil dimerization domain or an immunoglobulin light chain constant region 40. As shown, the domain 30 fused to the MHC α chain domain 10 is further fused to a hinge region 50 (optional) and Fc region 60 of an immunoglobulin chain. Alternatively, not shown, the MHC α and β chain domains 10 and 20 may be switched such that the MHC β chain domain is fused to the immunoglobulin heavy chain domains 50 and 60. The dimerization domains 30 and 40 promote the assembly of these two fusion constructs to form a heterodimeric structure which binds an MHC binding peptide 110 in the cleft formed by the MHC Class II binding domains of the α and β chains, 10 and 20. Optionally, flexible molecular linkers, not shown, are interposed between the MHC domains (10, 20) and the dimerization domains (30, 40), and/or between the dimerization domain 30 and the immunoglobulin hinge 50 or Fc region 60. The Fc regions 60 and 60′ of two of these heterodimeric MHC-immunoglobulin fusion proteins associate in the manner of an antibody to form a divalent MHC binding domain fusion protein construct. Horizontal lines between the Fc regions 60 and 60′ represent disulfide bridges between the immunoglobulin heavy chain domains.

FIG. 3. This figure is a schematic representation of one embodiment of a decavalent MHC binding domain fusion protein construct of the invention. Here, an extracellular or peptide binding domain of an MHC Class II α chain 10 is joined to either a first coiled-coil dimerization domain or an IgM immunoglobulin heavy chain C_{H}1 (Cμ1) constant domain 30, an extracellular or peptide binding domain of an MHC Class II K chain 20 is joined to either a second coiled-coil dimerization domain or an IgM immunoglobulin light chain constant region 40, and these two fusion constructs assemble to form a heterodimeric molecule which binds an MHC binding peptide 110 in the cleft formed by the MHC Class II binding domains of the ax and β chains, 10 and 20. As shown, the domain 30 fused to the MHC α chain domain 10 is further fused to an IgM Fc domain (C_{H}2, C_{H}3, C_{H}4) 60. Alternatively, not shown, the MHC a and D chain domains 10 and 20 may be switched such that the MHC D chain domains are fused to the immunoglobulin heavy chain domains 60. The Fc regions 60 and 60′ of two heterodimeric MHC-immunoglobulin fusion proteins associate in the manner of a single IgM subunit to form a divalent MHC-IGM fusion structure joined by a disulfide bond. Five of these divalent MHC-IgM fusion subunits assemble to form a characteristic IgM pentamer, joined by disulfide bonds 90 between IgM subunits and including a J-chain peptide 100, and resulting in a decavalent MHC-IgN fusion structure. Optionally, flexible molecular linkers, not shown, are interposed between the MHC domains (10, 20) and the coiled-coil or IgM dimerization domains (30, 40), and/or between the dimerization domains (30) and the IgM Fc domains (60).

FIG. 4. This figure is a schematic representation of one embodiment of a tetravalent MHC binding domain fusion protein construct of the invention. Here, an extracellular or peptide binding domain of an MHC Class II α chain 10 is joined to a first dimerization domain 30, an extracellular or peptide binding domain of an MHC Class II β chain 20 is joined to a second dimerization domain 40, and these two fusion constructs assemble to form a heterodimeric molecule which binds an MHC binding peptide 110 in the cleft formed by the MHC Class II binding domains of the α and β chains, 10 and 20. As shown, the domain 30 fused to the MHC a chain domain 10 is further fused to a ligand tag 70 which binds to anti-ligand 80. Alternatively, not shown, the MHC α and β chain domains 10 and 20 may be switched such that the MHC P chain domain is fused to the ligand tag 70. As shown, each anti-ligand binds four ligand moieties, and the MHC binding domain fusion protein complex is tetravalent. Optionally, flexible molecular linkers, not shown, are interposed between the MHC domains (10, 20) and the dimerization domains (30, 40), and/or between the dimerization domain 30 and the ligand tag 70.

FIG. 5. This figure is a schematic representation of one embodiment of a multimeric MHC binding domain conjugate of the invention. Here, an extracellular or peptide binding domain of a first MHC chain (α or β) 10 and an extracellular or peptide binding domain of a second MHC chain (o or a) 20 assemble to form a heterodimeric molecule which binds an MHC binding peptide 110 in the cleft formed by the MHC binding domains of the α and β chains, 10 and 20. A conjugating moiety 200 conjugates, covalently or non-covalently, one of the MHC chains 10 to a carrier 300.

FIG. 6. This figure is a schematic representation of one embodiment of a multimeric MHC binding domain conjugate of the invention. Here, an extracellular or peptide binding domain of an MUC α chain 10 is joined to a first dimerization domain 30, an extracellular or peptide binding domain of an NMC β chain 20 is joined to a second dimerization domain 40, and these two fusion constructs assemble to form a heterodimeric molecule which binds an MHC binding peptide 110 in the cleft formed by the MHC binding domains of the α and β chains, 10 and 20. As shown, the domain 30 fused to the MHC α chain domain 10 is bound, covalently or non-covalently, to a conjugating moiety 200 which is bound, covalently or non-covalently, to a carrier 300. Here, the carrier 300 is shown as a dendrimer. Alternatively, not shown, the MHC a and β chain domains 10 and 20 may be switched such that the MHC β chain domain is bound to the conjugating moiety 200. Optionally, flexible molecular linkers, not shown, are interposed between the MHC domains (10, 20) and the dimerization domains (30, 40), and/or between the dimerization domain 30 and the conjugating moiety 200, and/or between the conjugating moiety 200 and the carrier 300.

FIG. 7. This figure is a schematic representation of one embodiment of a multimeric MHC binding domain conjugate of the invention. Here, an extracellular or peptide binding domain of an MHC α chain 10 is joined to a first dimerization domain 30, an extracellular or peptide binding domain of an MHC β chain 20 is joined to a second dimerization domain 40, and these two fusion constructs assemble to form a heterodimeric molecule which binds an MHC binding peptide 110 in the cleft formed by the MHC binding domains of the α and β chains, 10 and 20. As shown, the domain 30 fused to the MHC α chain domain 10 is further fused to a ligand tag 70 which binds to anti-ligand 80, which is bound to the surface of a carrier 300. Alternatively, not shown, the MHC a: and β chain domains 10 and 20 may be switched such that the MHC β chain domain is fused to the ligand tag 70. Optionally, flexible molecular linkers, not shown, are interposed between the MHC domains (10, 20) and the dimerization domains (30, 40), and/or between the dimerization domain 30 and the ligand tag 70, and/or between the anti-ligand 80 and the carrier 300.

FIG. 8. This figure graphically presents the results of experiments demonstrating the assembly and secretion of recombinant HLA-DR2 fusion proteins by Pichia pastoris. Expression of DR2 fusion proteins (DRα-Fos and DRβ-Jun) were examined by sandwich ELISA of cell culture supernatants (top graph) or cell culture lysates (bottom graph) using a mAb (L243) specific for the DR2 αβ heterodimer for capture, and a polyclonal DR antiserum for detection. Binding of the secondary antibody was quantitated with a peroxidase conjugated anti-rabbit IgG antiserum, with ABTS as the peroxidase substrate and detection at 405 nm. Results are from cells transfected with: DRα-Fos only, open squares; DRβ-Jun only, solid circles; and both DRα-Fos and DRβ-Jun, open circles.

FIG. 9. This figure presents the results of experiments demonstrating the specificity of peptide binding to recombinant HLA-DR2 (rDR2) fusion proteins. Peptide binding was examined using a biotinylated MBP(85-99) peptide (“MBP”) that was previously shown to bind with high affinity to detergent soluble DR2. rDR2-MBP complexes were captured on an ELISA plate using a DR specific mAb (L243) and DR bound biotinylated MBP was quantitated using peroxidase labeled streptavidin, with ABTS as the peroxidase substrate and detection at 405 nm. The top graph shows the effect of rDR2 concentration on peptide binding with: 2 pIM biotinylated MBP peptide, open circles; 2 μM biotinylated MBP peptide with 100 μM unbiotinylated MBP as a competitor, solid triangles; and no peptide, solid squares. The same ELISA assay was used with 200 nM rDR2 and 2 μM biotinylated MBP to demonstrate binding specificity. The bottom graph shows the effect of varying concentrations of competitor peptides on biotinylated MBP peptide binding to rDR2 fusion proteins: unbiotinylated MBP competitor, open squares; Val89→Asp MBP competitor, closed circles.

FIG. 10. This figure presents the results of experiments demonstrating the kinetics of peptide binding to recombinant HLA-DR2 fusion proteins (rDR2). The kinetics of peptide binding were compared for rDR2 and for detergent soluble DR2 purified from an EBV transformed B cell line. The DR2 proteins (200 nM) were incubated with biotinylated MBP peptide (2 μM) at 37° C. for different periods of time; the amount of DR bound peptide was examined by ELISA using a DR specific antibody for capture and streptavidin-peroxidase for quantification of bound peptide, with ABTS as the peroxidase substrate and detection at 405 nm. The graph shows biotinylated MBP peptide binding over time for: recombinant DR2 fusions, open squares; detergent solubilized B cell DR2 molecules, closed triangles.

### DETAILED DESCRIPTION OF THE INVENTION

I. Definitions.

In order to more clearly and concisely describe and point out the subject matter of the claimed invention, the following definitions are provided for specific terms which are used in the following description and the claims appended hereto.

As used herein, the term “Major Histocompatibility Complex” and the abbreviation “MHC” means the complex of genes, found in all vertebrates, which function in signaling between lymphocytes and antigen presenting cells in normal immune responses by binding peptides and presenting them for possible recognition by T cell receptors (TCRs). MHC molecules bind peptides in an intracellular processing compartment and present these peptides on the surface of antigen presenting cells to T cells. The human MIC region, also referred to as HLA, is found on chromosome six and includes the Class I region (including the Class I a genes HLA-A, BLA-B and HLA-C) and the Class II region (including the subregions for Class II α and β genes DP, DQ and DR).

As used herein the term “MHC Class I” or “Class I” refers to the human Major Histocompatibility Complex Class I proteins, binding peptides, or genes. Within the MHC Class I region are found the HLA-A, HLA-B and HLA-C subregions. As used herein, the term “MHC Class I molecule” means a covalently or non-covalently joined complex of an MHC Class I a chain and a β₂-microglobulin chain.

As used herein, the term “MHC Class II” or “Class II” refers to the human Major Histocompatibility Complex Class II proteins, binding peptides, or genes. Within the MRC Class II region are found the DP, DQ and DR subregions for Class II α chain and β chain genes (i.e., DPα, DPβ, DQα, DQβ, DRα, and DRβ). As used herein, the term “MHC Class II molecule” means a covalently or non-covalently joined complex of an MHC Class II α chain and an MHC Class II β chain.

As used herein the term “MHC Class I α chain” means a naturally occurring polypeptide, or one encoded by an artificially mutated a gene, essentially corresponding to at least the α₁ and α₂ domains of one of the gene products of an MHC Class I a gene (e.g. HLA-A, HLA-B or HLA-C gene). As C-terminal transmembrane and cytoplasmic portions of the α chain are not necessary for membrane binding in the present invention, they may be omitted while retaining biological activity. In addition, the term “MHC Class I α chain” is intended to include variants with and without the usual glycosylation of the α₂ domain. The term is particularly intended to embrace all allelic variants of the Class I α genes, as well as any equivalents, including those which may be produced synthetically or recombinantly by, for example, site-directed mutagenesis of a naturally occurring variant. An MHC Class I α chain may also be referred to herein as an “MHC Class I heavy chain.”

As used herein the term “Class I β chain” or “β₂-microglobulin” means a naturally occurring polypeptide, or one encoded by an artificially mutated β₂-microglobulin gene, essentially corresponding to the gene product of a β₂-microglobulin gene. The term is particularly intended to embrace all allelic variants of β₂-microglobulin, as well as any equivalents, including those which may be produced synthetically of recombinantly by, for example, site-directed mutagenesis of a naturally occurring variant. When the term “MHC β chain” is used without specifying whether the chain is Class I or Class II, the term is intended to include β₂-microglobulin as well as MHC Class II β chains. A β₂-microglobulin or MHC Class β chain may also be referred to herein as an “MHC Class I light chain.”

As used herein, the term “MHC Class II α chain” means a naturally occurring polypeptide, or one encoded by an artificially mutated a gene, essentially corresponding to at least the α, and α₂ extracellular domains of one of the gene products of an MHC Class II a gene (e.g., a DP, DQ or DR a gene). As the C-terminal transmembrane and cytoplasmic portions of the α chain are not necessary for antigenic peptide binding in the present invention, they may be omitted while retaining biological activity. In addition, the term “MHC Class II α chain” is intended to include variants with and without the usual glycosylation of the α, and α₂ domains. The term is particularly intended to embrace all allelic variants of the Class II α genes, as well as any equivalents which may be produced synthetically or recombinantly by, for example, site-directed mutagenesis of a naturally occurring variant.

As used herein, the term “MHC Class II β chain” means a naturally occurring polypeptide, or one encoded by an artificially mutated β gene, essentially corresponding to at least the β₁ and β₂ extracellular domain of one of the gene products of an MHC Class III β gene (e.g., DP, DQ or DR β gene). As the C-terminal transmembrane and cytoplasmic portions of the β chain are not necessary for antigenic peptide binding in the present invention, they may be omitted while retaining biological activity. In addition, the term “MHC Class II β chain” is intended to include variants with and without the usual glycosylation of the β₁ domain. The term is particularly intended to embrace all allelic variants of the Class II β genes, as well as any equivalents which may be produced synthetically or recombinantly by, for example, site-directed mutagenesis of a naturally occurring variant.

As used herein the term “MHC binding domain” means an MHC Class I binding domain and/or an MHC Class II binding domain.

As used herein the term “MFC Class I binding domain” refers to the region of an MHC Class I molecule which is necessary for binding an antigenic peptide. An NMC Class I binding domain is formed primarily by the α₁ and α₂ domains of the MHC Class I ax chain. Although the α₃ domain of the α chain and β₂-microglobulin are not essential parts of the binding domain, they are believed to be important in stabilizing the over-all structure of the MHC Class I molecule and, therefore, an MHC Class I binding domain of the present invention preferably includes these regions. An MHC Class I binding domain may also be essentially defined as the extracellular domain of an MHC Class I molecule, distinguishing it from the transmembrane and cytoplasmic domains, although it is likely that some portion of the extracellular domain may be omitted while retaining biological activity.

As used herein, the term “MHC Class II binding domain” refers to the region of an MHC Class II molecule which is necessary for binding an antigenic peptide. An MHC Class II binding domain is formed primarily by the α₁ and β₁ domains of the MHC Class II α and β chains and, therefore, an MHC Class II binding domain minimally includes these regions. The α₂ and β₂ domains of these proteins, however, are also believed to be important to stabilizing the over-all structure of the MHC binding cleft and, therefore, an MHC Class II binding domain of the present invention preferably includes these regions. An MHC Class II binding domain may also be essentially defined as the extracellular domain of an MHC Class II molecule, distinguishing it from the transmembrane and cytoplasmic domains, although it is likely that some portion of the extracellular domain may be omitted while retaining biological activity.

As used herein the term “MHC binding peptide” or “binding peptide” means an MHC Class I binding peptide and/or an MHC Class II binding peptide.

As used herein the term “IC Class I binding peptide” means a polypeptide which is capable of selectively binding within the binding cleft formed by a specified MHC Class I molecule to form a Class I MHC/peptide complex. An MHC Class I binding peptide may be a processed self or non-self peptide or may be a synthetic peptide. For Class I MHC/peptide complexes, the binding peptides are typically 8-10 amino acid residues in length, although longer and shorter ones may be effective.

As used herein, the term “MHC Class II binding peptide” means a polypeptide which is capable of selectively binding within the binding cleft formed by the α and β chains of a specified TMC Class II molecule to form a Class II MHC/peptide complex. An MIC Class II binding peptide may be a processed self or non-self peptide or may be a synthetic peptide. For Class II MHC/peptide complexes, the binding peptides are typically 10-25 amino acids in length, and more typically 13-18 residues in length, although longer and shorter ones may be effective.

As used herein, the term “MHC/peptide complex” means a covalently or non-covalently joined ternary complex of either (a) the binding domain of an SIC Class I molecule and an MHC Class I binding peptide which binds to that MHC Class I binding domain or (b) the binding domain of an MHC Class II molecule and an MHC Class II binding peptide which binds to that MHC Class II binding domain.

As used herein, the term “multimeric Major Histocompatibility Complex binding domain conjugate” or “multimeric MHC binding domain conjugate” means a conjugate of a multiplicity of MHC binding domains directly or indirectly joined, bound (covalently or noncovalently), attached, adsorbed, or otherwise conjugated to a carrier. The MHC binding domains may be, but need not be, part of the monovalent or multivalent MHC fusion proteins of the invention.

As used herein, the term “carrier” means a molecule, particle, composition, or other microscopic object to which may be conjugated, directly or indirectly, a multiplicity of MHC binding domains, so as to form a multimeric MHC binding domain conjugate. The MHC binding domains may be, but need not be, part of the monovalent or multivalent MHC fusion proteins of the invention.

As used herein, the term “dendrimer” refers to a branched polymer in which a multiplicity of core polymer branches extend outwards from a core or initiator molecule, each branch forming additional sub-branches as it extends further outward, thereby forming a structure in which the number of terminal branches exceeds the number of core polymer branches by at least a factor of two.

As used herein, the term “liposome” refers to an aqueous compartment enclosed by at least one bilayer of amphipathic molecules (e.g., phospholipids). The term liposome, as used herein, is intended to embrace unilamellar and multilamellar liposomes.

As used herein, the term “porous” means, with respect to a carrier, that there are a multiplicity of openings in the surface of the carrier which are in fluid communication with each other, and which define passages within said carrier of sufficient diameter to permit diffusion of low molecular weight compounds (e.g., less than 5 kDa) therethrough, but are of insufficient diameter to permit unimpeded movement of higher molecular weight compounds therethrough.

As used herein, the term “flexible molecular linker” or “linker” means a chemical moiety having a length equal to or greater than that of three carbon to carbon bonds and including at least one freely rotating bond along said length. Preferably, a flexible molecular linker is comprised of one or more amino acid residues but this need not be the case. In certain preferred embodiments, the flexible molecular linkers of the invention comprise at least three and, more preferably, at least seven amino acid residues.

As used herein the term “conjugating moiety” refers to a chemical moiety which directly or indirectly joins, binds (covalently or noncovalently), attaches, adsorbs, or otherwise conjugates an MHC binding domain, or a fusion protein comprising an MHC binding domain, and a carrier.

As used herein, the term “selectively binding” means capable of binding in the electro- and stereospecific manner of an antibody to antigen or ligand to receptor. With respect to an MHC binding peptide, selective binding entails the non-covalent binding of specific side chains of the peptide within the binding pockets present in the MHC binding domain in order to form an MHC/peptide complex (see, e.g., Brown et al., 1993; Stern et al., 1994).

As used herein, the term “substantially pure” means, with respect to the MHC binding peptides and various MHC binding domain fusion proteins of the invention, that these peptides or proteins are essentially free of other substances to an extent practical and appropriate for their intended use. In particular, the peptides and proteins are sufficiently pure and are sufficiently free from other biological constituents of their hosts cells so as to be useful in, for example, generating antibodies or producing pharmaceutical preparations. A substantially pure preparation of the peptides or proteins of the invention need not be absolutely free of all other proteins or cell components and, for purposes of administration, may be relatively dilute. One of ordinary skill in the art may produce such substantially pure preparations by application or serial application of well-known methods including, but not limited to, HPLC, affinity chromatography or electrophoretic separation. The substantially pure preparations of the invention may also comprise other active ingredients and, therefore, the percentage by weight of the MHC binding peptides and/or various MHC binding domain fusion proteins of the invention may be reduced in such a preparation.

As used herein, the term “particulate” describes a structure which extends in three dimensions and defines a minimal surface area and a minimal volume, and which includes at least one surface capable of being conjugated to a multiplicity of MHC binding domains in a substantially two dimensional array. The term “particulate” is intended to embrace carriers which are generally spherical, ellipsoidal, rod-shaped, globular, or polyhedral.

As used herein, the term “minimal surface” means, with respect to a carrier, the surface area of the smallest continuous surface which defines a volume which may contain the carrier. As used herein, the term “minimal volume” means the volume contained within a minimal surface.

II. Preferred Embodiments

A. Monovalent and Multivalent MHC Binding Domain Fusion Proteins

In one aspect, the present invention depends, in part, upon the discovery that fusion proteins, comprising MHC Class II binding domains and coiled-coil and/or immunoglobulin constant domains, may be recombinantly produced, and that these fusion proteins may form heterodimers which include biologically functional MHC Class II binding domains in monovalent or multivalent fusion proteins. In particular, it is disclosed that (1) heterodimeric MHC Class II binding domains, including those of certain MHC Class II molecules which previously could not be produced as empty, soluble, stable heterodimers, may be produced using fusion proteins incorporating dimerization domains, and (2) heterodimeric MEC Class II binding domains, with or without dimerization domains, may be produced in the form of multivalent fusion protein constructs by incorporating them as fusions in multivalent immunoglobulin or ligand/anti-ligand structures. Of particular importance, is the surprising result that the MHC Class II binding domains of these fusion proteins retain their biological activity despite the functional requirement for highly specific tertiary and quaternary structural interactions within and between the α and β chains of the MHC molecule, and despite the substitution of relatively large, relatively hydrophilic fusion domains for the natural, hydrophobic transmembrane domains of the MHC Class II proteins.

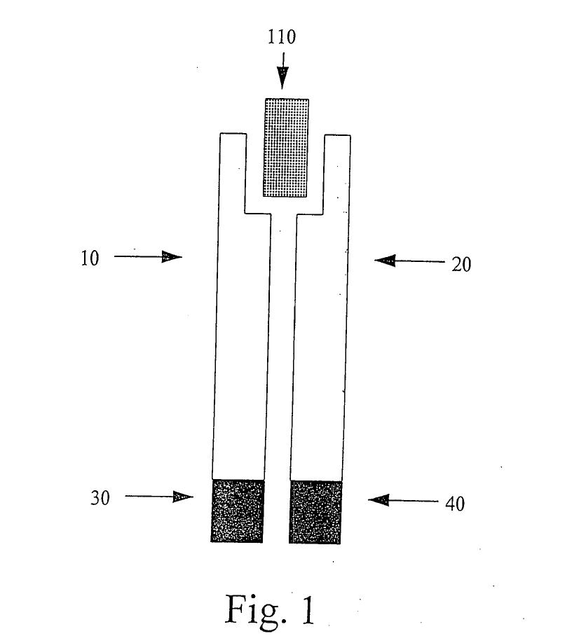

Thus, in a first series of embodiments, the present invention provides for the production of fusion proteins of MHC Class II α and β chain proteins in which substantially all of the C-terminal transmembrane and cytoplasmic domains have been replaced by coiled-coil dimerization domains and, optionally, interposing linker sequences. FIG. 1 schematically illustrates such a monovalent MHC Class II binding domain fusion protein. At least the peptide binding domain, and preferably the entire extracellular domain, of an MHC Class II β chain 10 may be fused to a first dimerization domain 30 (e.g., a leucine zipper domain or an immunoglobulin Fab constant domain). Similarly, at least the peptide binding domain, and preferably the entire extracellular domain, of an MHC Class II β chain 20 may be fused to a second dimerization domain 40 (e.g., a leucine zipper domain or an immunoglobulin Fab constant domain). The dimerization domains (30 and 40) associate in solution to promote formation of a heterodimeric fusion protein in which the MHC Class II α and β chain components (10 and 20) stably associate to form a biologically active MHC Class II binding domain which is capable of binding, or being “loaded” with, an MHC binding peptide 110 so as to form a stable MHC/peptide complex which can selectively bind to cognate T cell receptors and/or selectively activate T cell clones bearing cognate TCRs. Optionally, flexible molecular linkers may be interposed between the MHC components (10 and 20) and dimerization domains (30 and 40) so as to better approximate the normal distance between the MHC components and their natural MHC transmembrane domains, and/or to provide for free rotation between the MHC components and the dimerization domains such that the geometry of the association between dimerization domains does not constrain or interfere with the geometry of association of the MHC binding domains.

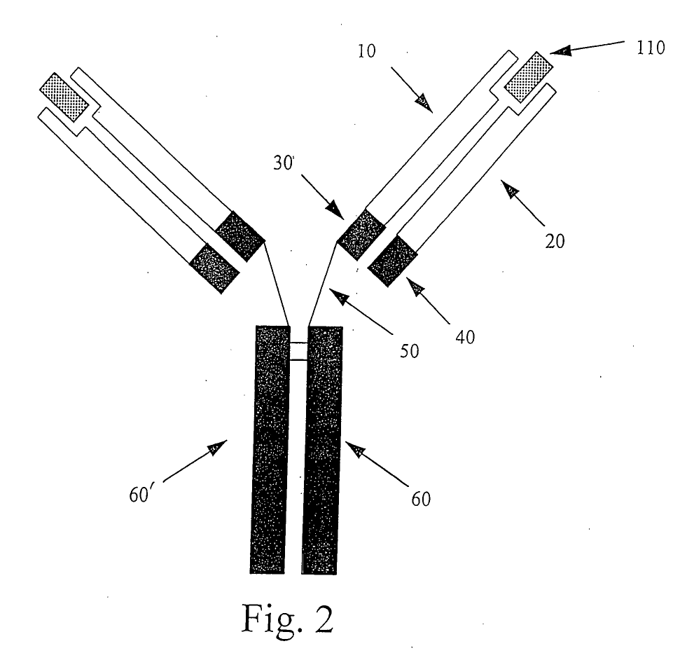

In another series of embodiments, the present invention provides for the production of divalent fusion proteins of MHC Class II α and β chain proteins in which substantially all of the C-terminal transmembrane and cytoplasmic domains have been replaced by immunoglobulin constant chain domains and, optionally, interposing linker sequences and/or coiled-coil dimerization domains. The immunoglobulin constant domains are chosen so as to promote the formation of divalent antibody-like molecules bearing two MHC Class II binding domains and, optionally, to promote certain effector functions (e.g., complement activation, cell binding). FIG. 2 schematically illustrates such a divalent MHC Class II binding domain fusion protein. At least the peptide binding domain, and preferably the entire extracellular domain, of an MHC Class II α chain 10 may be fused to a first dimerization domain 30 (e.g., a leucine zipper domain or an immunoglobulin Fab constant domain). Similarly, at least the peptide binding domain, and preferably the entire extracellular domain, of an MHC Class II chain 20 may be fused to a second dimerization domain 40 (e.g., a leucine zipper domain or an immunoglobulin Fab constant domain). The dimerization domains (30 and 40) associate in solution to promote formation of a heterodimeric fusion protein in which the MHC Class II a and P components (10 and 20) stably associate to form a biologically active MHC Class II binding domain which is capable of binding, or being “loaded” with, an MHC binding peptide 110 so as to form a stable MHC/peptide complex which can selectively bind to cognate T cell receptors and/or selectively activate T cell clones bearing cognate TCRs. As noted above, however, some MHC Class II molecules (e.g., KLA-DR1, HLA-DR4) can be expressed by the method of Stem and Wiley (1992) as stable, soluble heterodimers without their transmembrane and cytoplasmic domains. For such molecules, coiled-coil dimerization domains are not necessary to the formation of heterodimeric MHC binding domains and, therefore, may be omitted entirely from these embodiments or may be replaced by Fab constant domains (i.e., heavy chain C_{H}1 domains or light chain CL domains). Next, one of the two MHC fusion proteins further comprises an immunoglobulin Fc region 60, with or without an interposing immunoglobulin hinge region 50 appropriate to the Fc region (IgA, IgD and IgG molecules include hinge regions; IgE and IgM molecules do not). Preferably, it is the MHC Class II α chain fusion protein which is fused to the immunoglobulin heavy chain Fc region because the MHC Class II α chains are less variable than the β chains and, therefore, such an α chain fusion protein can be used with a number of different MHC Class II D chain fusion proteins to form a variety of different divalent molecules with different HLA specificity. It should, however, be noted that there is no reason that the D chain construct can not include the immunoglobulin Fc regions. Finally, flexible molecular linkers may be optionally interposed between the MHC components (10 and 20), dimerization domains (30 and 40), and/or immunoglobulin components (50 and/or 60) so as to better approximate the normal distance between the MHC components and their natural MHC transmembrane domains, and/or to provide for free rotation between the MHC components, the dimerization domains, and/or the immunoglobulin domains such that the geometry of the association between any pair of dimerizing components does not constrain or interfere with the geometry of association or dimerization of the others. As shown in FIG. 2, the immunoglobulin heavy chain Fc regions 60 and 60′ of two such MHC Class II fusion proteins associate and form a divalent structure, with one or more disulfide linkages between chains, analogous to the structure of natural antibodies.

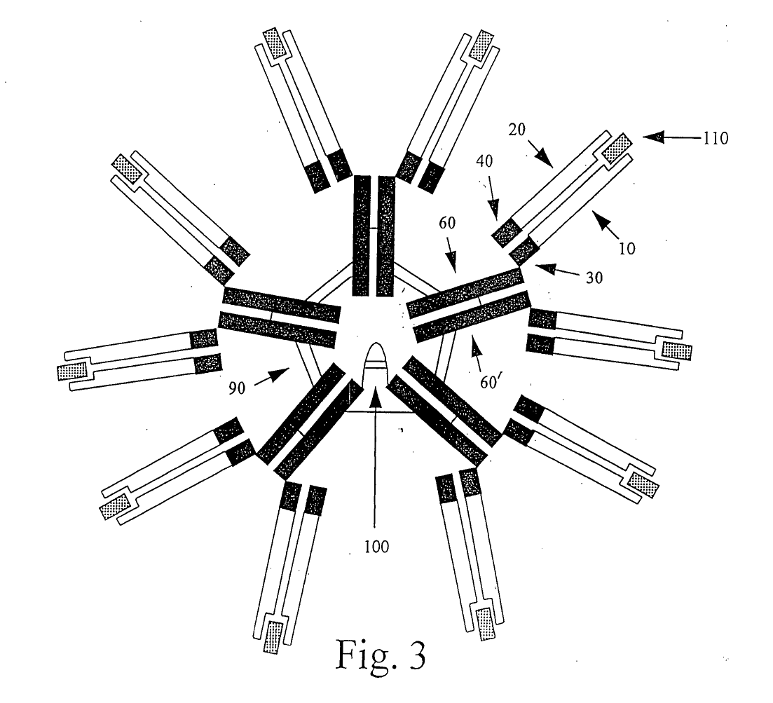

In another series of embodiments, the present invention provides for the production of decavalent fusion proteins of MHC Class II α and β chain proteins in which substantially all of the C-terminal transmembrane and cytoplasmic domains have been replaced by IgM immunoglobulin constant chain domains and, optionally, interposing linker sequences and/or coiled-coil dimerization domains. These embodiments are essentially the same as those described immediately above except that (1) the immunoglobulin constant domains are specifically chosen to be IgM chains, which form divalent subunits which are then assembled into decavalent pentamers, and (2) that the cells producing these MHC-IgM fusions are cotransfected with a J-chain gene in order to facilitate the assembly and secretion of IgM molecules (Matsuuchi et al., 1986). FIG. 3 schematically illustrates such a decavalent MHC Class II fusion protein. As before, at least the peptide binding domains, and preferably the entire extracellular domains, of MHC Class II α 10 and β 20 chains may be fused to dimerization domains 30 and 40 (e.g., a leucine zipper domain or an immunoglobulin Fab constant domain). The dimerization domains (30 and 40) associate in solution to promote formation of a heterodimeric fusion protein in which the MHC Class II a and p components (10 and 20) stably associate to form a biologically active MHC Class II binding domain which is capable of binding, or being “loaded” with, an MFC binding peptide 110. Again, as noted above, some MHC Class II molecules (e.g., HLA-DR1, HLA-DR4) can be expressed by the method of Stem and Wiley (1992) as stable, soluble heterodimers without their transmembrane and cytoplasmic domains and, therefore, for such molecules, coiled-coil dimerization domains may be omitted entirely or may be replaced by Fab constant domains (i.e., heavy chain C_{H}1 domains or light chain CL domains). Next, as above, either the α or β chain construct further comprises an immunoglobulin Fc region 60 which, in these embodiments, is an IgM Fc region (C_{H}2, C_{H}3, C_{H}4). Finally, as before, flexible molecular linkers may be optionally interposed between the MHC components (10 and 20), dimerization domains (30 and 40), and/or IgM Fc components (60) so as to better approximate the normal distance between the MHC components and their natural MHC transmembrane domains, and/or to provide for free rotation between the MHC components, the dimerization domains, and/or the immunoglobulin domains. As shown in FIG. 3, the immunoglobulin heavy chain Fc regions 60 and 60′ of two such MHC-IgM fusion proteins are associated to form a divalent structure with one or more disulfide linkages between chains. These divalent subunits, however, will further associate to form a multimer with one or more disulfide bonds 90 between divalent subunits. In the presence of the J-chain protein 100, IgM subunits are assembled into decavalent pentamers as shown in FIG. 3, analogous to naturally occurring IgM pentamers.

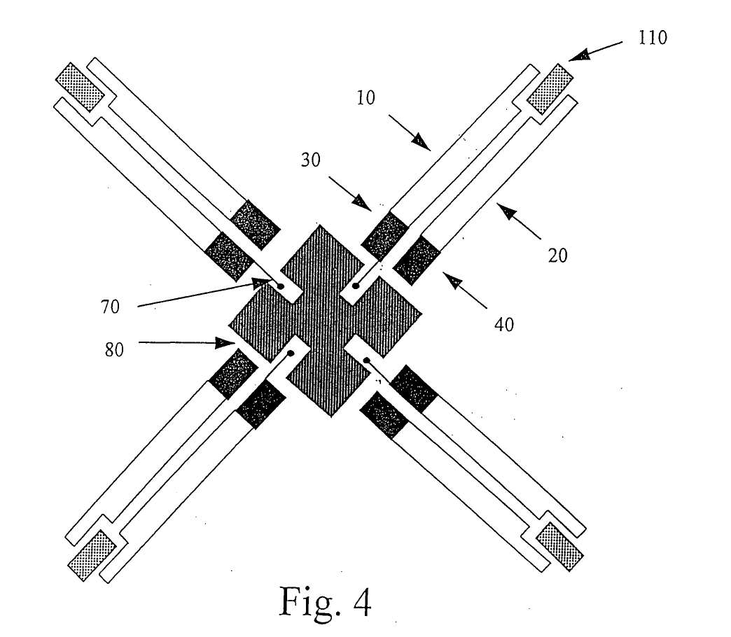

In another series of embodiments, the present invention provides for the production of tetravalent fusion proteins of MHC Class II a and D chain binding domains in which substantially all of the C-terminal transmembrane and cytoplasmic domains have been replaced by dimerization domains and, optionally, interposing linker sequences, and in which a C-terminal ligand “tag” sequence allows a multiplicity of MHC-tag fusions to bind to an anti-ligand and form a multivalent MHC binding domain fusion protein complex. The ligand tag sequence may be any sequence for which an anti-ligand is available, or any sequence which facilitates the addition of a ligand to the tag. For example, the tag sequence may be a poly-His sequence, which may serve as a ligand for a Ni⁺-bearing anti-ligand. Alternatively, the tag sequence may be the epitope of an antibody, and the anti-ligand may be that antibody. In a preferred embodiment, the tag is a recognition sequence which may be biotinylated by biotin ligase, and the anti-ligand may be avidin or streptavidin. FIG. 4 schematically illustrates a tetravalent MHC binding domain fusion protein complex in which a biotinylated tag serves as the ligand, and avidin or streptavidin serves as the anti-ligand. As in previous embodiments, at least the peptide binding domain, and preferably the entire extracellular domain, of MHC Class II α chains 10 and β chains 20 may be fused to dimerization domains (30 and 40) (e.g., a leucine zipper domain or an immunoglobulin Fab constant domain). The dimerization domains (30 and 40) associate in solution to promote formation of a heterodimeric fusion protein in which the MHC Class II α and β components (10 and 20) stably associate to form a biologically active MHC Class II binding domain which is capable of binding, or being “loaded” with, an MHC binding peptide 110. In addition, a biotin ligase recognition sequence or “tag” is fused to the C-terminus of at least one of the MHC binding domain fusion chains. This sequence tag may be biotinylated by enzymes within the cells which produce these MHC binding domain fusion proteins, or may be subsequently biotinylated in vitro. The biotinylated tag 70 can be used to cause the monovalent MHC binding domain fusion proteins to bind to avidin (or streptavidin) 80. As each avidin (or streptavidin) molecule is capable of binding up to four biotin moieties, an MHC-biotin/(strept)avidin fusion protein complex can be produced which is tetravalent (with lower valencies at lower molar ratios of biotin:(strept)avidin). As before, flexible molecular linkers may optionally be interposed between the MHC components (10 and 20), the dimerization domains (30 and 40) and/or the biotin sequence tag so as to better approximate the normal distance between the MHC components and their natural MHC transmembrane domains, and/or to provide for free rotation between the MHC components, the dimerization domains, and/or biotinylated tag. As will be apparent to one of ordinary skill in the art, a great variety of other ligand tags and anti-ligands may be employed instead of biotin/(strept)avidin to produce similar multivalent MHC binding domain fusion protein complexes.

In each of the foregoing embodiments, the MHC binding peptide 110 may be covalently joined to either the MHC Class II α or β components (10 and 20) with a flexible molecular linker (not shown in the Figures). Preferably, such flexible molecular linkers are polypeptide sequences of 10-20 amino acid residues, more preferably 12-18 amino acid residues. When the flexible molecular linkers are polypeptides, the MHC binding peptide, linker and MHC Class II α or β components may all be expressed as a single fusion protein, further comprising dimerization domains toward the C-terminus.

In connection with each of the above-described embodiments, the present invention provides (a) isolated nucleic acid sequences encoding such fusion proteins; (b) vectors for transiently or stably transfecting host cells with these nucleic acids; (c) host cells transformed with these sequences or vectors; (d) methods for producing the fusion proteins employing these sequences, vectors and host cells; and (e) the substantially purified fusion proteins themselves. In addition, the present invention provides for a number of utilities for these products and processes including, but not limited to, the treatment of allergic and autoimmune diseases, the detection and/or isolation of T cells with defined MHC/peptide specificities, and the selective activation, anergization, or killing of T cells with defined MHC/peptide specificities.

1. Choice of MHC Components for Monovalent and Multivalent MFC Fusion Proteins

The methods and products of the present invention may be practiced with any mammalian MHC Class II proteins. Primarily, however, it is anticipated that the present invention will have greatest utility in the diagnosis and treatment of human disease and, therefore, the MHC Class II proteins are preferably human HLA Class II proteins. Thus, for example, the present invention may be practiced with either of the known HLA-DRA alleles (DRA*0101 and DRA*0102), any of the approximately 160 known HLA-DRB alleles (including at least 137 known HLA-DRB1 alleles), any of the approximately 15 known HLA-DQA1 alleles, any of the approximately 25 known HLA-DQB1 alleles, any of the approximately 8 known HLA-DPA1 alleles, or any of the approximately 65 known HLA-DPB1 alleles. A compilation of known human HLA Class II nucleotide sequences has been published by Marsh and Bodmer (1995), and a compilation of known HLA Class II nucleotide and amino acid sequences is available via electronic transfer from the EMBL Data Library, Cambridge, UK (request “HiELP HLA” by e-mail to “netserv@ebi.ac.uk”). All of these sequences are not, therefore, reproduced herein. In addition, all sequence nomenclature used herein conforms to that used in Marsh and Bodmer (1995), and in Bodmer et al. (1995).

Embodiments employing coiled-coil dimerization domains are particularly preferred for use with those MHC Class II binding domains which, without their transmembrane and cytoplasmic domains, do not form stable heterodimers in solution. For these proteins, the coiled-coil domains add stability to the heterodimer while allowing for the production of soluble, non-aggregated proteins. Amongst these are the BLA-DR2 serotypes (e.g., those encoded by DRA and DRB1*15 or DRB1*16 alleles), HLA-DQ8 (encoded by, for example, the DQA1*0301 and DQB1*0302 alleles), and HLA-DQ2 (encoded by, for example, DQA1*0501 and DQB1*0201 alleles). Nonetheless, coiled-coil dimerization domains may be employed with any of the human MHC Class II binding domains, including those which have previouslybeen successfully expressed as stable, soluble heterodimers without their transmembrane domains (e.g., DR1 and DR4).

2. Choice of MHC Class II Binding Domain Splice Points

In accordance with the present invention, splice points for the MHC components of the MHC Class II binding domain fusion proteins must be chosen so as to include sufficient N-terminal sequence for proper formation of an MHC binding domain while excluding most if not all of the C-terminal transmembrane and cytoplasmic domains of the MHC chains. As is well known in the art, the MHC Class II α and β chains are each characterized by two N-terminal, globular, extracellular domains (α1 and α2, or β1 and β2), followed by a short loop or connecting peptide, a hydrophobic transmembrane domain, and a C-terminal hydrophilic cytoplasmic domain. The binding cleft of the MHC Class II molecules is formed primarily by the interaction of the α and β1 domains in the heterodimer and, therefore, these domains must minimally be included in the fusion proteins of the present invention. The α2 and β2 domains, however, are also preferably included because they may aid in stabilizing the MHC binding domain, are believed to be involved in the formation of dimers of the MHC chains, and are believed to be involved in CD4 receptor binding.

Thus, in preferred embodiments, the splice points for the MHC Class II fusion peptides are chosen to be in the regions approximately between the ends of the α2 or β2 domains and the beginnings of the transmembrane domains. For most MHC Class II α chains, this corresponds to approximately amino acid residue positions 180-200, and for most β chains this corresponds to approximately amino acid residue positions 185-205. For example, for the HLA-DR ac chains encoded by the DRA alleles (DRA*0101 or DRA*0102), the transmembrane domains essentially begin after the Glu residue at position 191 or the Asn residue at position 192. For the HLA-DR β chains encoded by the DR1 subtype DRB1*01 (e.g., DRB1*0101) alleles and the DR2 subtype DRB1*15 and DRB1*16 alleles (e.g., DRB1*1501), the transmembrane domains essentially begin after the Lys residue at position 198 or the Met residue at position 199. Similarly, for the DQ2 and DQ8 subtypes, the HLA-DQA1 transmembrane domains essentially begin after the Glu residue at position 195 or the Thr residue at position 196, and the HLA-DQB1 transmembrane domains essentially begin after the Lys residue at position 200 or the Met residue at position 201. For some allelic variants, of course, there may be amino acid insertions or deletions prior to these sites which alter the residue numbering. The connecting peptide and transmembrane regions of the MHC Class II α and β chains are not, however, highly polymorphic and, indeed, appear invariant for all known DRA and DRB alleles (see Marsh and Bodmer, 1995). Therefore, working with any given MHC Class II α and β chains, one of ordinary skill in the art can easily identify the transmembrane domains both by homology to the above-described alleles, and by their essential hydrophobic nature.

Although not preferred, it is acceptable that the fusion proteins of the present invention include several residues from the transmembrane domain or that several residues of the α₂ or β₂ domains be omitted. For example, the inclusion of 1-5 residues of the transmembrane domain may be included in the present invention and still yield a soluble fusion protein, but this is not preferred. Similarly, the omission of, for example, 1-5 residues from the α₂ or β₂ domains may not result in structural alterations which disrupt MHC peptide binding, heterodimer formation, or T cell interactions. Indeed, if replaced by suitable residues which conserve the over-all structure of the MHC molecule, larger portions of the α₂ and β₂ structural domains may be omitted in accordance with the present invention (e.g., replacing portions of the Class II α₂ or β₂ domains with equivalent portions of the Class I α₃ or β₂-microglobulin proteins). Thus, recognizing that one may also include or omit all or part of the 5-7 amino acid loop or connecting peptide which naturally joins the α₂ and β₂ domains to their respective transmembrane domains, one may produce fusion proteins in which the splice point is anywhere from approximately residues 180-200 of the α chain and approximately residues 185-205 of the (3 chain, with larger C-terminal omissions tolerated if appropriate replacement sequences are provided. Finally, 1-5 residues may be omitted or substituted at the N-terminus of an MHC Class II α or β chain binding domain, although this is not recommended.

In preferred embodiments, however, the splice points are chosen to be within the loop or connecting peptide sequence near the N-terminal end of the transmembrane domain. In the most preferred embodiments, the entire extracellular domains of the MHC Class II α and β chains are included in the MHC fusion proteins of the invention.

3. MHC Class II Binding Peptide Fusions

In connection with any of the foregoing embodiments, one may also create a fusion protein in which an MHC binding peptide is covalently joined to the N-terminus of either the α or β chain such that the binding peptide is capable of selectively binding within the binding domain formed by the given MHC Class II α and β chains. Preferably, the MHC binding peptide is joined to the N-terminus of the β chain because, when the α and β chains associate to form a heterodimeric MHC molecule, the N-terminus of the (chain is more accessible than the N-terminus of the α chain. In addition, the β chains of MHC Class II molecules are more polymorphic than the α chains and, therefore, the specificity of an MHC binding domain is more dependent upon which β chain is included in the molecule.

The MHC binding peptide is preferably linked to the MHC binding domain using a flexible molecular linker, as described below. In preferred embodiments, the flexible molecular linker is a polypeptide sequence of approximately 10-20 amino acid residues, more preferably 12-18 amino acid residues, which joins the binding peptide and MHC binding domains by standard polypeptide linkages to form a larger fusion protein which may be encoded by a single nucleic acid construct and expressed as a single fusion protein. In addition, it is preferred that the amino acids be chosen from the relatively small residues (e.g., alanine, glycine, serine, leucine, isoleucine, valine) in order to minimize the potential for steric hindrance.

As noted above, the MHC binding peptide is chosen such that it is capable of selectively binding to the MHC molecule to which it is attached. Thousands of combinations of MHC binding peptides and MHC molecules are known in the art and can be identified by standard methods (see, e.g., Chicz et al., 1993). Of particular interest are those pairs of MHC binding peptides and MHC molecules which are implicated in diseases, including infections and autoimmune diseases. For example, specific MHC binding peptides derived from the human myelin basic protein (e.g., residues 85-99, 84-102 and 148-162) and particular MHC alleles (e.g., BLA-DR2 or DRA/DRB1*1501) have been implicated in the development of multiple sclerosis. Therefore, in one preferred embodiment, monovalent or multivalent MHC Class II binding domain fusion proteins are produced in which an immunogenic myelin basic protein (MBP) peptide is covalently joined by a polypeptide linker sequence to the N-terminus of the peptide binding domain of an HLA-DRB1*1501 protein, and this fusion is covalently joined, with or without an interposing flexible molecular linker, to a dimerization domain. Such a fusion protein may then be dimerized with a corresponding HLA-DRA ax chain fusion protein such that the MBP peptide binds in the cleft formed by association of the MHC Class II ax and β chain binding domains. Similarly, certain residues of the human desmoglein 3 protein (e.g., residues 78-93, 97-111, 190-204, 206-220, 251-265, 512-526 and 762-786) and certain MHC alleles (e.g., HLA-DR4 or DRA/DRB1*0402, and HLA-DQ1 or DQA/DQB1*05032) have been implicated in the development of pemphigus vulgaris (see, e.g., WO 96/27387). For other autoimmune diseases, including rheumatoid arthritis (RA) and systemic lupus erythematosus (SLE), immunodominant self peptides may be identified which selectively bind to particular MHC Class II molecules. For each of these, monovalent or multivalent MFC Class II binding domain fusion proteins may be produced, having the autoimmunogenic MHC binding peptides covalently joined to the MHC binding domains, and these may be used, as further described below, in identifying, sorting, selecting or targeting autoreactive T cells, or in tolerizing or anergizing the immune response to the autoantigens.

4. Choice of Linker Domains

In accordance with the present invention, MHC binding domain fusion proteins may be produced which optionally include flexible molecular linkers which covalently join, as described above, (1) MHC binding domains to dimerization domains; (2) dimerization domains to immunoglobulin Fc domains or ligand tag domains; or (3) MHC binding peptides to MHC binding domains. Appropriate linkers include, but are not limited to, short polypeptide chains which can be encoded with the MHC domains, dimerization domains, immunoglobulin domains, and/or tag domains in recombinant DNA constructs. More generally, however, appropriate linkers include any relatively small (e.g., <2 kDa, preferably <1 kDa) organic chemical moieties which are flexible because they include at least one single bond located between their termini and about which there is free rotation. Thus, for example, bifunctional molecules (e.g., an α,ω-dicarboxylic acid or an α,ω-diamine) of a lower alkyl chain may be employed, and such flexible molecular linkers may be reacted with the C-termini of the MHC components and the N-termini of the coiled-coil, immunoglobulin or ligand tag components (or with reactive groups of the amino acid side chains of any of these). Many other cross-linking agents, of course, are well known in the art and may be employed as substantial equivalents.

Preferably, however, the flexible molecular linkers of the present invention comprise a series of amino acid residues which can be encoded in a fusion gene construct. For example, a linker of 1-15 generally small amino acid residues (e.g., alanine, glycine, serine, leucine, isoleucine, valine), optionally including one or more hydrophilic residues (e.g., aspartate, glutamate, lysine), may be employed as a linker. For linkers between MHC binding domains and dimerization domains, the length of the linker may be chosen so as to maintain, approximately, the spacing naturally found between the MHC binding domains and the transmembrane domains of the MHC proteins and, therefore, the length of the linker may depend upon whether some or all of the naturally occurring loop or connecting residues between the binding domains and transmembrane domains have been included or omitted. In addition, as will be apparent to one of skill in the art, linker sequences may be particularly chosen so as to introduce specific proteinase cleavage sites in the fusion protein or, for ease of recombinant DNA manipulations, to introduce specific restriction endonuclease sites into the recombinant construct. Thus, for example, one may include the naturally occurring 5-7 amino acid loop or connecting peptides of an MHC molecule and also include a 5-7 amino acid linker. Alternatively, thelincluded portion of the loop or connecting peptide may be varied, the linker length may be varied, or the loop peptide and/or linker may be omitted entirely. Using standard techniques of site-directed mutagenesis, or restriction and ligation of recombinant constructs with a variety of different endonucleases, one of ordinary skill in the art can easily produce many variations on the fusion protein constructs and, as described below, rapidly test them for cognate TCR binding and/or T cell activation. Thus, although some presently preferred embodiments employ the entire extracellular domains of the MHC molecules joined by particular linkers to dimerization, immunoglobulin or ligand tag domains, the invention is not limited to such embodiments.

5. Choice of Dimerization Domains

Coiled-coils are common structural features of dimeric proteins in which two α-helical polypeptides (“coils”) are twisted (“coiled”) about each other to form a larger quaternary structure or “coiled-coil” (see, e.g., Hu et al., 1990; Oas and Endow, 1994). Indeed, the transmembrane regions of HLA-DR α and β chains are thought to be a helices that assemble as a coiled-coil within the hydrophobic environment of the cell membrane (Cosson and Bonifacino, 1992). Other coiled-coils, however, are hydrophilic and may be found in secreted, cytosolic and nuclear proteins. For example, “leucine zippers” are coiled-coil domains which are present in a large number of DNA binding proteins and which may mediate either homodimer or heterodimer formation (see, e.g., Ferré-D'Amaré et al., 1993; O'Shea et al, 1989; O'Shea et al., 1991). In addition, several researchers have now designed artificial coiled-coil domains, including pairs of basic and acidic amphipathic helices and artificial leucine zippers, which have been expressed and assembled in recombinant homodimeric and heterodimeric proteins (see, e.g., Pack and Pluckthun, 1992; Chang et al., 1994).

In preferred embodiments of the present invention, the dimerization domains are leucine zipper domains. These leucine zippers are characterized by at least 4 and, preferably, at least 5-7 leucine residues that are spaced periodically at approximately every seventh residue (heptad repeat), with each heptad repeat contributing two turns of the α-helix (3.5 residues/turn). The leucine residues appear to have a special function in coiled-coil dimerization, and form part of the hydrophobic interface between the two α-helices in the coiled-coil. The 40 amino acid leucine zipper domains of the proteins Fos and Jun are preferred examples of leucine-zipper dimerization domains. These domains each have five leucine residues spaced-exactly every seventh residue with a number of hydrophilic residues in the intervening positions (the Fos sequence includes three additional leucines which do not fall in the heptad repeat pattern and which, therefore, are assumed not to contribute to heterodimer formation). Modifications of these domains, or even completely artificial sequences, which maintain the over-all helical nature of these sequences (e.g., which do not include proline or hairpin turns), which preserve the over-all hydrophilicity of the helixes, and which preserve the approximate heptad repeat of leucine residues, may also be employed in accordance with the invention. (Note that the scHLX amphipathic helix of Pack and Pluckthun (1992) was tested in a recombinant LA-DR2 usion construct but did not lead to successful heterodimer formation.)

Finally, as noted above, some MHC Class II molecules (e.g., HLA-DR1, HLA-DR4) have been successfully produced as soluble, stable heterodimers without their transmembrane domains (see, e.g., Stern and Wiley, 1992). For such molecules, or for others which assemble with moderate stability, a coiled-coil dimerization domain may not be necessary. Thus, in some embodiments of the present invention, such domains may be omitted entirely or, alternatively, other domains which promote dimerization may be substituted. In particular, it is contemplated that the constant domains of the Fab fragments of immunoglobulins (i.e., the CHI and CL domains) may be employed as heterodimer-forming dimerization domains.

6. Choice of Immunoglobulin Domains for MHC Binding Domain Fusion Proteins

Human immunoglobulins are divided into five broad classes (IgA, IgD, IgE, IgG and IgM) and any of these may be employed in the MHC-immunoglobulin constructs of the present invention. The basic structures of these molecules are extremely well characterized disulfide-linked homodimers of heavy and light chain heterodimers. Thus, the basic immunoglobulin unit resembles the protein of FIG. 2 in which a pair of heavy chains, corresponding to elements 10, 30, 50 and 60, are disulfide linked toeach other, and the N-terminal end of each heavy chain is associated with a light chain, corresponding to elements 20 and 40. The light chain and the portion of the heavy chain associated with it, corresponding to elements 10, 20, 30 and 40, are referred to as the Fab fragment. The portions of the two heavy chains which are closely associated with each other, 60 and 60′, are referred to as the Fc fragment. In some classes of immunoglobulins (i.e., IgA, IgD, and IgG), there is a hinge region 50 between the Fab and Fc fragments. Finally, within both the light and heavy immunoglobulin chains, there are regions of great variability and regions of great constancy. Thus, the immunoglobulin light chains include a variable domain V_{L}, corresponding essentially to element 20 of FIG. 2 (but not to scale), and a constant domain C_{L}, corresponding essentially to element 40 (but not to scale). Similarly, each heavy chain includes an N-terminal variable domain V_{H}, corresponding essentially to element 10 (but not to scale), and three or four constant domains C_{H}1 through C_{H}4, corresponding essentially to elements 30, 50 and 60 (but not to scale). (See, generally, Kuby, 1994). The immunoglobulin constant domains, astheir name implies, are relatively invariant in the human population. On the basis of differences in their constant regions, light chains are broadly classified as either κ or λ and, in humans, λ chains are further divided into four subtypes. Similarly, differences in the constant regions of immunoglobulin heavy chains are the basis of the division of these molecules into five broad classes (α, δ, ε, γ and μ chains in IgA, IgD, IgE, IgG and IgM, respectively). Based on minor differences in amino acid sequences in humans, the γ chains have been further subdivided into four subclasses, and the α chains into two. The amino acid sequences of these various immunoglobulin light and heavy chain constant domains have long been known in the art (see, e.g., Kabat et al., 1979) and will not be reproduced here.

In some preferred embodiments, the MHC-immunoglobulin fusion proteins of the present invention include the Fc regions of either IgG (subtypes 1, 2 or 3) or IgM because these Fc domains are capable of activating the classical complement pathway and, therefore, are more useful in some of the therapeutic methods described below. For utilities in which complement activation is not desired or is irrelevant, however, any of the immunoglobulin isotypes may be employed. In addition, as shown in FIG. 3, the IgM isotypes are preferred in some embodiments because they can form pentamers of divalent MHC-IgM fusion proteins.

7. Expression Systems for MHC Binding Domain Fusion Proteins

The MHC binding domain fusion proteins of the present invention may be expressed in any standard protein expression system which allows for proper folding and secretion of the desired molecules, or which allows for their recovery as properly folded molecules from inclusion bodies. As a general matter, eukaryotic expression systems are preferred because they are most likely to produce a high yield of properly folded, glycosylated and disulfide-linked molecules. Mammalian cell lines, especially those which are well characterized for protein expression (e.g., CHO cells, COS cells) or those which are known to secrete properly folded, glycosylated and disulfide linked immunoglobulins (e.g., any mAb producing cell line), may be preferred for some uses. Generally, however, these cell lines express too little protein for therapeutic and commercial applications. Therefore, other eukaryotic expression systems, such as the Pichia pastoris yeast system, described below, may be preferred. In addition, preliminary results have shown high levels of expression of an MHC binding domain fusion protein in a Drosophila Schneider cell system. It is well within the abilities and discretion of the skilled artisan without undue experimentation, to choose an appropriate or favorite expression system.

Similarly, once a design (i.e., primary amino acid sequence) for the MHC binding domain fusion proteins of the present invention is chosen, one of ordinary skill in the art can easily design appropriate recombinant DNA constructs which will encode the desired proteins, taking into consideration such factors as codon biases in the chosen host, the need for secretion signal sequences in the host (e.g., an α-mating factor secretory signal for yeast expression), the introduction of proteinase cleavage sites within the signal sequence, and the like. These recombinant DNA constructs may be inserted in-frame into any of a number of expression vectors appropriate to the chosen host. The choice of an appropriate or favorite expression vector is, again, a matter well within the ability and discretion of the skilled practitioner. Preferably, of course, the expression vector will include a strong promoter to drive expression of the recombinant constructs and, optionally, a number of marker genes which will simplify the identification and/or selection of transformants.

B. Multimeric MHC Binding Domain Conjugates

In another aspect, the present invention depends, in part, upon the discovery that multimeric MHC binding domain conjugates comprising a multiplicity of MHC binding domains conjugated to a carrier may be produced, and that these multimeric conjugates have far greater avidity for their cognate TCRs, and far greater biological activity, than monovalent MHC binding domains, or even divalent or tetravalent MHC binding domain constructs. Without being bound to any particular theory of the invention, it is believed that a great increase in avidity of T cell binding and/or activation may be achieved by providing a multiplicity of MHC binding domains on a single carrier such that a substantially two-dimensional array of MHC binding domains may make contact with an area of a T cell membrane bearing a multiplicity of T cell receptors.

Thus, in one series of embodiments, the present invention provides for the production of multimeric MHC binding domain conjugates in which about 5-500 MHC binding domains, preferably about 10-200 MHC binding domains, and more preferably about 20-100 MHC binding domains, are conjugated to a single carrier. The carrier can be characterized as defining a minimal surface area and, preferably, the average density of the MHC binding domains on that surface is between about 4×10⁻³ to 20 MHC binding domains/nm²; more preferably about 4×10⁻2 to 20 MHC binding domains/nm², and most preferably about 0.4 to 20 MHC binding domains/nm².

Moreover, in preferred embodiments, the size and weight of the multimeric MHC binding conjugates are limited to aid in maintaining solubility, and to avoid possible complications caused by aggregation in vivo. Thus, it is preferred that the largest cross-sectional diameters of the MHC binding domain conjugates of the invention are less than about 1,000 nm, preferably less than about 500 nm, and more preferably less than about 100 nm. If perfectly spherical, such conjugates would define a minimal surface area of less than approximately 3.1×10⁶ nm², 7.9×10⁵ nm², and 3.1×10⁴ nm², respectively, and would define a minimal volume of 5.2×10⁸ nm³, 6.5×10⁷ nm³, and 5.2×10⁵ nm³. In the most preferred embodiments, as described below, the MHC binding domain conjugates have maximum diameters of about 5-40 nm. If perfectly spherical, such conjugates would define minimal surface areas of approxiamtely 78.5 to 5.0×10³ nn², and would define minimal volumes of 65.4 to 3.4×10⁴ nm³. In addition, it is preferred that the overall weights of the MHC binding domain conjugates are less than about 10,000 kDa, preferably less than about 5,000 kDa, and more preferably less than about 1,000 kDa. In the most preferred embodiments, as described below, the MHC binding domain conjugates have weights of about 200-500 kDa.

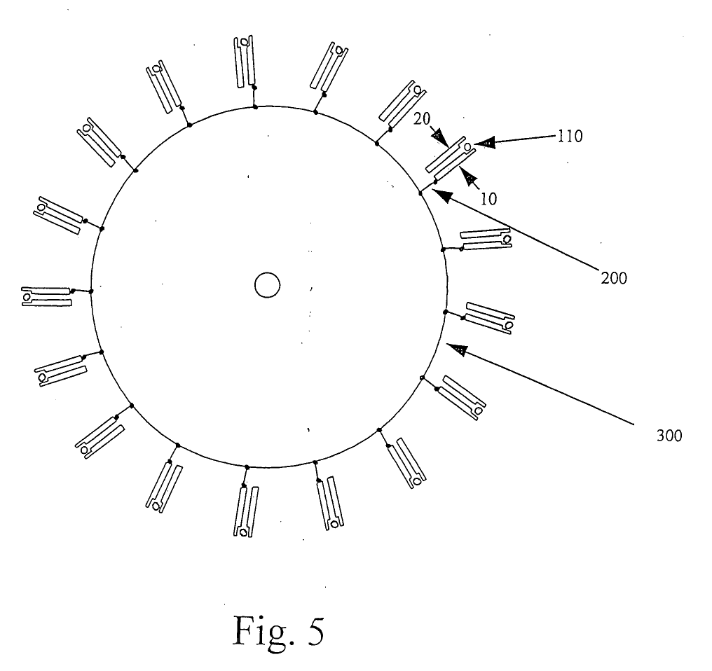

FIG. 5 schematically illustrates one embodiment of a multimeric MHC binding domain conjugate comprising a multiplicity of MHC binding domains conjugated to a carrier. Thus, the conjugate comprises at least the binding domains, and preferably the entire extracellular domains, of a multiplicity of MHC α chains 10 which are stably associated with at least the binding domains, and preferably the entire extraceilular domains, of MHC β chains 20 to form biologically active MHC binding domains which are capable of binding, or being “loaded” with, MHC binding peptides 110. In this figure, the MHC α chain 10 is shown as being conjugated to a carrier 300 by a conjugating moiety 200. Alternatively, however, the MHC β chain 20 may be conjugated to the carrier 300 by a conjugating moiety 200. In other embodiments, the conjugating moiety 200 may be omitted and one of the MHC chains (10 or 20) may be directly conjugated to the carrier 300. The MHC binding domains are conjugated to the carrier in an orientation which allows interaction of the MHC/peptide complexes with the TCRs on cognate T cells. As shown in FIG. 5, the carrier 300 is depicted as a substantially spherical particle, but this need not be the case.

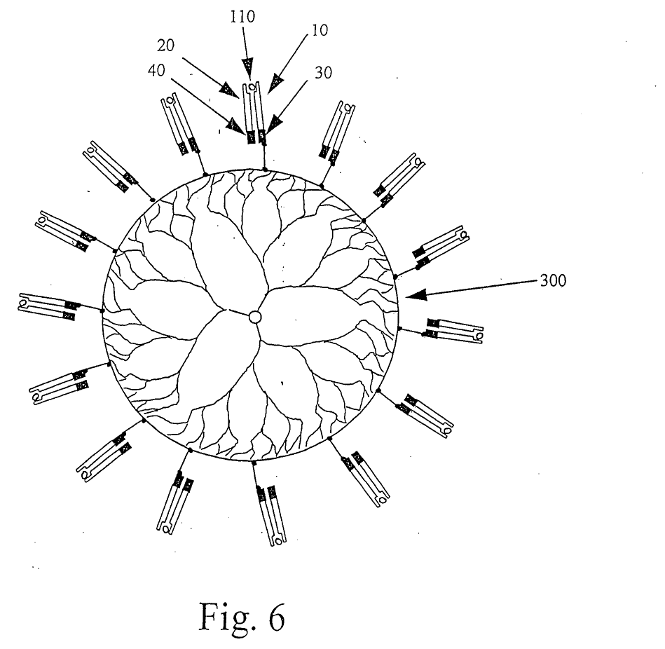

FIG. 6 schematically illustrates another embodiment of a multimeric MHC binding domain conjugate of the invention. In this figure, the conjugate comprises a multiplicity of MHC binding domain fusion proteins, such as those described above. Thus, in this embodiment, at least the peptide binding domains of a multiplicity of MHC α chains 10 have been joined to first dimerization domains 30, at least the peptide binding domains of a multiplicity of MHC β chains 20 have been joined to second dimerization domains 40, and these fusion proteins have assembled to form heterodimeric MHC binding domains which may bind MHC binding peptides 110. Flexible molecular linkers, not shown, optionally may be interposed between the MHC domains (10, 20) and the dimerization domains (30, 40). In this figure, the first dimerization domains 30 are shown as being conjugated to a carrier 300 by a conjugating moiety 200. Alternatively, however, the second dimerization domains 40 may be conjugated to the carrier 300 by a conjugating moiety 200. In other embodiments, the conjugating moiety 200 may be omitted and one of the dimerization domains (30 or 40) may be directly conjugated to the carrier 300. The MHC binding domain fusion proteins are conjugated to the carrier in an orientation which allows interaction of the MHC/peptide complexes with the TCRs on cognate T cells. As shown in FIG. 6, the carrier 300 is depicted as a substantially spherical branched polymer or dendrimer, but this need not be the case.

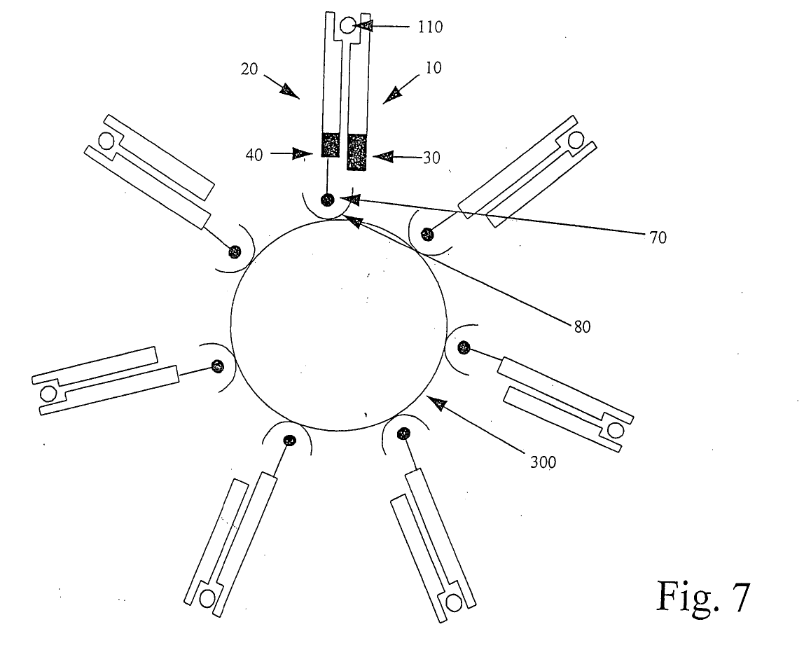

FIG. 7 schematically illustrates another embodiment of a multimeric MHC binding domain conjugate of the invention. In this figure, the conjugate comprises a multiplicity of MHC binding domain fusion proteins, such as those described above, and the numbered elements 10, 20, 30, 40 and 110 are as described in FIG. 6. In this embodiment, however, the conjugating moiety 200 of FIG. 6 has been replaced by two elements, 70 and 80. Thus, a first conjugating moiety 70 is bound, covalently or non-covalently, to the second dimerization domain 40, and the second conjugating moiety 80 is bound, covalently or non-covalently, to the carrier 300. For example, the first conjugating moiety 70 may be a biotin-tag, as described above, and the second conjugating moiety 80 may be avidin or streptavidin. Alternatively, and as described below, the conjugating moieties 70 and 80 may be any pair of molecules which are capable of binding to each other, covalently or non-covalently, so as to conjugate the MHC binding domains to the carrier.

In other embodiments, the various elements depicted in FIGS. 1-7 may be interchanged or mixed. Thus, for example, the monovalent MHC binding domain fusion protein of FIG. 1, the divalent MHC binding domain fusion protein of FIG. 2, the decavalent MEC binding domain fusion protein of FIG. 3, or the tetravalent MHC binding domain fusion protein of FIG. 4, may be conjugated by conjugating moieties as in FIG. 5 or 6, or by first and second conjugating moieties as in FIG. 7, to a particulate carrier as in FIG. 5 or 7, or to a branched polymer or dendrimer carrier as in FIG. 6. In addition, and as described herein, other embodiments not depicted in the figures may be employed.

1. Choice of MHC Components for Multimeric MHC Binding Domain Conjugates

In contrast to the previously described monovalent and multivalent MHC binding domain fusion proteins, in which MHC Class II binding domains were employed with dimerization domains to produce stable and soluble heterodimers of the fusion proteins, the multimeric MHC binding domain conjugates of the present invention may employ either Class I or Class II MHC binding domains. As before, essentially any mammalian MHC proteins may be employed but, as it is anticipated that the present invention will have greatest utility in the diagnosis and treatment of human disease, the MHC Class I and MHC Class. I proteins are preferably human MHC proteins:

Thus, any of the MHC binding domains which were described above for use in MHC binding domain fusion proteins may also be used in the production of multimeric-MHC binding domain conjugates. Similarly, the same MHC splice points, described above, may be employed to obtain the complete extracellular portion of MHC Class II α and β chains, or just the minimal portions of those chains which include the MHC binding domain.

Furthermore, any of the above-described monovalent or multivalent MHC binding domain fusion proteins may be employed as the MHC component in a multimeric MHC binding domain conjugate. Thus, for example, divalent MHC binding domain fusion proteins comprising at least the MHC binding domain of an MHC Class II molecule joined to immunoglobulin domains (with or without intervening coiled-coil dimerization domains or interposing flexible molecular linkers) may be conjugated to a carrier to produce a multimeric NHC binding domain conjugate of the invention. Similarly, the tetravalent and decavalent MHC binding domain fusion protein constructs described above may be conjugated to a carrier to produce a multimeric MHC binding domain conjugate. More simply, the monovalent MHC binding domain fusion proteins, employing coiled-coil or other dimerization domains as described above, may be conjugated to a carrier to produce a multimeric MHC binding domain conjugate.

In addition, however, multimeric MHC binding domain conjugates may be produced using MHC Class II binding domains which are free of exogenous coiled-coil or dimerization domains. Thus, for example, the extracellular or peptide binding domains of those MHC Class II molecules which are stable under physiological conditions without exogenous dimerization domains, such as those which may be produced by the methods of Stem and Wiley (1992), also may be conjugated to a carrier to produce a multimeric MHC binding domain conjugate of the invention. The MHC binding domains of the Class II HLA-DR1 and HLA-DR4 molecules may, for example, be produced in truncated form, and may be conjugated to a carrier without dimerization domains to aid in the stabilization of the heterodimer. The considerations in the choice of splice points for such truncated MHC Class II proteins are the same as those described above for the MHC binding domain fusion proteins and will not be repeated here.

Moreover, multimeric MHC binding domain conjugates may be produced in which the MHC binding domains are derived from MHC Class I as well as MHC Class II proteins. In particular, Class I MHC binding domains do not-require stabilization bydimerization domains because the light chains of these molecules (i.e., β₂-microglobulin) lack a transmembrane domain in nature, but are nonetheless able to stably associate with Class I α chains under physiological conditions. Therefore, MHC binding domain conjugates may be produced employing β₂ microglobulin in association with at least the peptide binding domain of an MHC Class I α chain conjugated to a carrier. The choice of splice points for MHC Class I α chains, like that of MHC Class II α chains, is within the ability of one of ordinary skill in the art. Specifically, however, the splice point is preferably chosen between residues at the C-terminus of the α₃ domain and residues at the N-terminus of the transmembrane domain (e.g., between about residues 273-283 of the mature HLA-A2 protein, preferably after the Pro residue at position 283 or the Ile residue at position 284). Preferably, the entire extracellular domain of an MHC Class I a: chain is employed.

2. Choice of Carriers for MHC Binding Domain Conjugates

The MHC binding domain conjugates of the present invention may be produced with any of a large variety of carriers including, but not limited to, particles, beads, branched polymers, dendrimers, or liposomes. The carriers must be capable of being conjugated, either directly or indirectly, to a multiplicity of MHC binding domains and, therefore, preferably comprise a multiplicity of reactive groups near the surface which can be used in conjugation reactions. Alternatively, however, the carrier may have a surface to which conjugating moieties may be adsorbed without chemical bond formation. Preferably the carrier is particulate, and generally spherical, ellipsoidal, rod-shaped, globular, or polyhedral in shape. Alternatively, however, the carrier may be of an irregular or branched shape. In preferred embodiments, the carrier is composed of material which is biodegradable and non-immunogenic. It is further preferred that the carrier have a net neutral or negative charge, in order to reduce non-specific binding to cell surfaces which, in general, bear a net negative charge.

As described above with respect to the MHC binding conjugates, the overall size and weight of the carriers are important considerations. Preferably, the carriers are microscopic or nanoscopic in size, both to enhance solubility, and to avoid possible complications caused by aggregation in vivo. Thus, it is preferred that the largest cross-sectional diameters of the carriers of the invention are less than about 1,000 nm, preferably less than about 500 nm, and more preferably less than about 100 nm. In the most preferred embodiments, as described below, carriers have maximum diameters of about 5-40 nm. Similarly, it is preferred that the overall weights of the carriers are less than about 10,000 kDa, preferably less than about 5,000 kDa, and more preferably less than about 1,000 kDa. In the most preferred embodiments, as described below, the carriers have weights of about 200-500 kDa.

(a) Microbead or Nanobead Carriers

In one series of embodiments, the present invention provides for the production of multimeric MHC binding domain conjugates in which a multiplicity of MHC binding domains are conjugated to a substantially spherical microbead or nanobead. The beads may be solid, hollow, or porous. For certain embodiments, in which it is desired to deliver a marker (e.g., a fluorescent agent) or therapeutic agent (e.g., a cytotoxin or lymphokine) to T cells bearing a particular TCR, it is preferred that the beads are porous.

Carrier beads can be formed from a wide range of materials. For example, beads may be composed of glass, silica, polyesters of hydroxy carboxylic acids, polyanhydrides of dicarboxylic acids, or copolymers of hydroxy carboxylic acids and dicarboxylic acids. More generally, the carrier beads may be composed of polyesters of straight chain or branched, substituted or unsubstituted, saturated or unsaturated, linear or cross-linked, alkanyl, haloalkyl, thioalkyl, aminoalkyl, aryl, aralkyl, alkenyl, aralkenyl, heteroaryl, or alkoxy hydroxy acids, or polyanhydrides of straight chain or branched, substituted or unsubstituted, saturated or unsaturated, linear or cross-linked, alkanyl, haloalkyl, thioalkyl, aminoalkyl, aryl, aralkyl, alkenyl, aralkenyl, heteroaryl, or alkoxy dicarboxylic acids. Carrier beads including mixtures of ester and anhydride bonds (e.g., copolymers of glycolic and sebacic acid) may also be employed. Thus, for example, carrier beads may comprise materials including polyglycolic acid polymers (PGA), polylactic acid polymers (PLA), polysebacic acid polymers (PSA), poly(lactic-co-glycolic) acid copolymers (PLGA), poly(lactic-co-sebacic) acid copolymers (PLSA), poly(glycolic-co-sebacic) acid copolymers (PGSA), etc. Other biocompatible, biodegradable polymers useful in the present invention include polymers or copolymers of caprolactones, carbonates, amides, amino acids, orthoesters, acetals, cyanoacrylates and degradable urethanes, as well as copolymers of these with straight chain or branched, substituted or unsubstituted, alkanyl, haloalkyl, thioalkyl, aminoalkyl, alkenyl, or aromatic hydroxy- or di-carboxylic acids. In addition, the biologically important amino acids with reactive side chain groups, such as lysine, arginine, aspartic acid, glutamic acid, serine, threonine, tyrosine and cysteine, or their enantiomers, may be included in copolymers with any of the aforementioned materials to provide reactive groups for conjugating to MHC binding domains or conjugating moieties. Currently preferred biodegradable materials include PLA, PGA, and PLGA polymers. See, generally, U.S. Pat. Nos. 1,995,970; 2,703,316; 2,758,987; 2,951,828; 2,676,945; 2,683,136 and 3,531,561. Biocompatible but non-biodegradable materials may also be used in the carrier beads of the invention. For example, non-biodegradable polymers of acrylates, ethylene-vinyl acetates, acyl substituted cellulose acetates, non-degradable urethanes, styrenes, vinyl chlorides, vinyl fluorides, vinyl imidazoles, chlorosulphonated olefins, ethylene oxide, vinyl alcohols, TEFLON® (DuPont, Wilmington, Del.), and nylons may be employed. See, generally, U.S. Pat. Nos. 2,609,347; 2,653,917; 2,659,935; 2,664,366; 2,664,367; and 2,846,407.

In currently preferred embodiments, the beads are composed of polystyrene, silica, PGA, PLA, PSA, PLGA, PLSA, or PGSA. Suitable beads which are currently available commercially include polystyrene beads such as FluoSpheres (Molecular Probes, Eugene, Oreg.), and silica beads such as Spherisorb™ (Phase Separation, North Wales, UK).

In currently preferred embodiments, carrier beads are employed having an average diameter of about 10-400 nm, more preferably 20-100 nm, and most preferably about 40 nm.

(b) Branched Polymer Carriers

In another series of embodiments, the present invention provides for the production of conjugates wherein a multiplicity of MHC binding domains are conjugated to a branched polymer. Branched polymers are preferable to linear polymers because they have numerous chain-ends or termini which can be functionalized and, therefore, can be conjugated to a multiplicity of MHC binding domains, either directly or indirectly through conjugating moieties.

Preferably, the branched polymer carriers of the invention are dendrimers. Dendrimers, also known as arborols, cascade molecules, dendritic polymers, or fractal polymers, are highly branched macromolecules in which the branches emanate from a central core. In one method of dendrimer production, dendrimers are synthesized outward from a core molecule by sequential addition of layers of monomers. The first round of dendrimer synthesis adds a single layer or “generation” of monomers to the core, with each monomer having at least one free, reactive terminus. Each subsequent round of polymerization results in the expansion of the dendrimer by one layer and increases the number of free, reactive termini. This process can be repeated numerous times to produce dendrimers of desired diameter or mass. As the density of the branches increases, the outermost branches arrange themselves in the form of a sphere surrounding a lower density core. See, for example, U.S. Pat. No. 5,338,532. In addition, by varying the shape of the core molecules, dendrimers may be produced in rod-shaped, disk-like, and comb-like forms. The resulting dendrimers may possess an arbitrarily large number of free, reactive termini, to which a multiplicity of MHC binding domains may be conjugated, either directly or indirectly. FIG. 6 provides a schematic depiction of a multimeric MHC binding domain conjugate comprising a dendrimer carrier 300.

In preferred embodiments, the dendrimer comprises a polyamidoamine; a polyamidoalcohol; a polyalkyleneimine such as polypropyleneimine or polyethyleneimine; a polyalkylene such as polystyrene or polyethylene; a polyether; a polythioether; a polyphosphonium; a polysiloxane; a polyamide; or a polyaryl polymer. Dendrimers have also been prepared from amino acids (e.g., polylysine). Suitable dendrimers which are currently available commercially include polyamidoamine dendrimers such as Starburst™ dendrimers (Dendritech, Midland, Mich.). The Starburst™ dendrimers terminate in either amine groups or carboxymethyl groups which may be used, with or without further modification, and with or without interposing conjugating moieties, to conjugate MHC binding domains to the surface of these carriers. Preferably, dendrimers are employed which terminate in carboxyl or other negatively charged reactive groups.

The different “generations” of dendrimers differ in weight, size and number of terminal reactive groups. For example, Generation 1 polyamidoamine Starburstm dendrimers have a molecular weight of ˜1.0 kDa, a diameter of ˜1.6 nm, and 6 terminal groups; Generation 2 have a molecular weight of ˜2.4 kDa, a diameter of ˜2.2 nm, and 12 terminal groups; Generation 3 have a molecular weight of ˜5.1 kDa, a diameter of ˜3.1 nm, and 24 terminal groups; Generation 4 have a molecular weight of ˜10.6 kDa, a diameter of ˜4.0 nm, and 48 terminal groups; Generation 5 have a molecular weight of ˜21.6 kDa, a diameter of ˜5.3 nm, and 96 terminal groups; Generation 6 have a molecular weight of ˜43.5 kDa, a diameter of ˜6.7 nm, and 192 terminal groups; Generation 7 have a molecular weight of ˜87.2 kDa, a diameter of ˜8.0 nm, and 384 terminal groups; Generation 8 have a molecular weight of ˜174.8 kDa, a diameter of −9.2 nm, and 768 terminal groups; Generation 9 have a molecular weight of −349.9 kDa, a diameter of ˜10.5 nm, and 1536 terminal groups; and Generation 10 have a molecular weight of ˜700 kDa, a diameter of ˜12.4 nm, and 3072 terminal groups (Roberts et al., 1996).

Non-dendrimer branched polymers may also be employed in the invention, and may be produced from the same general classes of materials as dendrimers. The synthesis of such branched polymers is also well known in the art. As used herein, a “branched polymer” means a polymer having at least 5 termini, preferably at least 10 termini, and more preferably 20-500 termini, formed by branching of a carbon and/or heteroatom backbone.

(c) Liposome Carriers

In another series of embodiments, the present invention provides for the production of multimeric MHC binding domain conjugates in which a multiplicity of MHC binding domains are conjugated to the outer surface of a liposome. Liposomes, also called lipid vesicles, are aqueous compartments enclosed by lipid membranes, and are typically formed by suspending a suitable lipid in an aqueous medium, and shaking, extruding, or sonicating the mixture to yield a dispersion of vesicles. Various forms of liposomes, including unilamellar vesicles and multilamellar vesicles, may be used in the present invention.

Liposomes may be prepared from a variety of lipid materials including, but not limited to, lipids of phosphatidyl choline, phosphatidyl serine, phosphatidyl inositol, phosphatidyl glycerol, phosphatidyl ethanolamine, phosphatidic acid, dicetyl phosphate, monosialoganglioside, polyethylene glycol, stearyl armine, ovolecithin and cholesterol, as well as mixtures of these in varying stoichiometries. Liposomes, as used herein, may also be formed from non-lipid amphipathic molecules, such as block copolymers of poly(oxyethylene-b-isoprene-b-oxyethylene) and the like. In preferred embodiments, the liposomes are preparedfrom lipids that will form negatively charged liposomes, such as those produced from phosphatidyl serine, dicetyl phosphate, and dimyristoyl phosphatidic acid.

The surfaces of liposomes may also be modified to reduce immunogenicity or to provide convenient reactive groups for conjugation. For example, sialic acid or other carbohydrates, or polyethylene glycol or other alkyl or alkenyl polymers, may be attached to the surface of a liposome to reduce immunogenicity. Alternatively, liposomes may be produced bearing a conjugating moiety such as biotin by inclusion of a small molar percentage of, for example, biotin-X-dipalmitoylphosphatidylethanolamine (Molecular Probes, Eugene, Oreg.) in the liposome.

3. Means of Conjugating MHC Binding Domains to a Carrier

A great variety of means, well known in the art, may be used to conjugate MHC binding domains to carriers to produce the MIC binding domain conjugates of the invention. These methods include any standard chemistries which do not destroy or severely limit the biological activity of the MHC binding domains, and which allow for a sufficient number of MHC binding domains to be conjugated to the carrier an orientation which allows for interaction of the MHC binding domain with a cognate T cell receptor. Generally, methods are preferred which conjugate the C-terminal regions of an MHC binding domain, or the C-terminal regions of an MHC binding domain fusion protein, to the carrier. The exact chemistries will, of course, depend upon the nature of the carrier material, the presence or absence of C-terminal fusions to the MHC binding domain, and/or the presence or absence of conjugating moieties.

In one series of embodiments, the MHC binding domains are bound to the carrier via a covalent chemical bond. For example, a reactive group or moiety near the C-terminus of the MHC cc or β chain (e.g., the C-terminal carboxyl group, or a hydroxyl, thiol, or amine group from an amino acid side chain) may be conjugated directly to a reactive group or moiety on the surface of the carrier (e.g., a hydroxyl or carboxyl group of a PLA or PGA polymer, a terminal amine or carboxyl group of a dendrimer, or a hydroxyl, carboxyl or phosphate group of a phospholipid) by direct chemical reaction. Alternatively, there may be a conjugating moiety which covalently conjugates to both the MHC binding domains and the carrier, thereby linking them together.

In some preferred embodiments, reactive carboxyl groups on the surface of a carrier may be joined to free amines (e.g., from Lys residues) on SIC binding domains, or MHC binding domain fusion proteins, by reacting them with, for example, 1-ethyl-3-[3,9-dimethyl aminopropyl] carbodiimide hydrochloride (EDC) or N-hydroxysuccinimide ester (NHS). Similarly, the same chemistry may be used to conjugate free amines on the surface of a carrier with free carboxyls (e.g., from the C-terminus, or from Asp or Glu residues) on MEC binding domains, or MHC binding domain fusion proteins. Alternatively, free amine groups on the surface of a carrier may be covalently bound to MHC binding domains, or MHC binding domain fusion proteins, using sulfo-SIAB chemistry, essentially as described by Arano et al. (1991).

In another series of embodiments, a non-covalent bond between a ligand bound to the MHC binding domain and an anti-ligand attached to the carrier may conjugate the NMC binding domains to the carrier. For example, as described above, a biotin ligase recognition sequence tag may be joined to the C-terminus of either (or both) of the MHC α or β chain binding domains, or to the C-terminus of an MHC binding domain fusion protein, and this tag may be biotinylated by biotin ligase. The biotin may then serve as a ligand to non-covalently conjugate the MHC binding domain to avidin or streptavidin which is adsorbed or otherwise bound to the surface of the carrier as an anti-ligand. Alternatively, if the MHC binding domains are fused to an immunoglobulin domain bearing an Fc region, as described above, the Fc domain may act as a ligand and protein A, either covalently or non-covalently bound to the surface of the carrier, may serve as the anti-ligand to non-covalently conjugate the MHC binding domain to the carrier. Other means are well known in the art which may be employed to non-covalently conjugate MHC binding domains to carriers, including metal ion chelation techniques (e.g., using a poly-His tag at the C-terminus of the MHC binding domain or MHC binding domain fusion proteins, and a Ni⁺-coated carrier), and these methods may be substituted for those described here.

4. Accessory Molecules Associated with MHC Binding Domain Conjugates

The MHC binding domain fusion proteins and conjugates of the present invention may also be associated with, or bound to, various accessory molecules or moieties which are suitable to particular utilities. For example, the fusion proteins or conjugates may be associated with, or bound to, molecules or moieties including cytotoxins (e.g., genistein, ricin, diphtheria toxins, Pseudomonas toxins, the Fas ligand, and radioactive isotopes) for killing T cells, or to T cell-modulating molecules (such as the B7-1, B7-2, LFA-3, CD40 or I-CAM proteins) for activating or anergizing T cells. In addition, the MHC binding domain fusion proteins and conjugates may be associated with, or bound to, various molecules or moieties which are useful for detecting the presence of the fusion proteins or conjugates, such as radioactive or fluorescent labels.

For MHC binding domain fusion proteins, such accessory molecules or moieties are preferably attached to the C-terminal region of the fusion protein or at some other point which is not expected to interfere with its ability to bind its cognate TCR (e.g., along an Fc domain, dimerization domain or flexible molecular linker). For MHC binding domain conjugates, the accessory molecules may be similar attached to the MHC binding domain or a fusion protein component (e.g., dimerization domains), to flexible molecular linkers or conjugating moieties, or to the carrier. For MHC binding domain conjugates in which the carrier is hollow (e.g., a liposome or hollow bead) or porous (e.g., a dendrimer or porous bead), an accessory molecule or moiety may be included within the interior or pores of the carrier. Inclusion within the, interior of a carrier is particularly preferred for cytotoxic agents which may exert their effect after the MHC binding domain conjugate is endocytosed within a T cell. For accessory molecules which exert T cell modulatory effects (e.g., B7-1, B7-2, and CD40, which are co-stimulatory molecules which aid in the activation of naive T cells) or accessory molecules which may promote adhesion of MHC binding domain conjugates to T cells (e.g., LFA-3 or I-CAM), the accessory molecule is preferably bound to the exterior of a carrier such as a bead, dendrimer, or liposome.

Accessory molecules may be bound to MHC binding domain conjugates by standard chemical techniques known in the art, including those described above for binding MHC binding domains, or MHC binding domain fusion proteins, to carriers. Accessory molecules may be associated within porous carriers, or included within hollow carriers, by standard techniques which are known in the art.

III. Uses for MHC Binding Domain Fusion Proteins and Conjugates

In one aspect, the present invention provides a method for detecting and/or isolating T cells of a defined MHC/peptide complex specificity comprising contacting a population of T cells with monovalent, multivalent or multimeric MHC: binding domain fusion proteins or conjugates of the invention, as described above, which are loaded with a particular MHC binding peptide and which, therefore, define a particular MHC/peptide complex. The activation or proliferation of the T cells may then be determined and used, with an appropriate control, as an indication of whether the T cell population includes T cells specific for the defined MHC/peptide complex. Alternatively, the monovalent, multivalent or multimeric MHC binding domain fusion proteins and conjugates of the invention, having a defined specificity, may be immobilized on a substrate and a population of T cells may be contacted with the immobilized MHC binding domains. After allowing a period of time for the binding, if any, of T cells specific for the defined MHC/peptide complex, unbound cells may be washed away and the presence or absence of bound T cells may be used as an indication of whether the T cell population includes T cells specific for the defined MHC/peptide complex. In another embodiment, the monovalent, multivalent or multimeric MHC binding domain fusion proteins and conjugates of the invention may be contacted with a T cell population and, after allowing a period of time for the binding, if any, of the MHC binding domains to T cells specific for the defined MHC/peptide complex, unbound fusion proteins or conjugates may be washed away and the presence or absence of bound fusion proteins or conjugates may be used as an indication of whether the T cell population includes T cells specific for the defined MHC/peptide complex. In all such embodiments, the labeling of the T cells, fusion proteins or conjugates, or complexes of the MHC/peptide complex with a reactive T cell receptor with fluorescent, radioactive or other markers is preferred to simplify detection. In particular, fluorescent labels may be used in conjunction with FACS (fluorescence-activated cell sorting) techniques to isolate a desired subpopulation of T cells with a defined MHC/peptide specificity.

In a particularly preferred embodiment, T cells which are reactive to a specific, defined MHC/peptide complex are detected and isolated as described above, and are then used, preferably after proliferation in vitro, for adoptive immunotherapy. Thus, a population of T cells may be obtained from a host (either the subject to be treated or a syngeneic donor). T cells which are reactive for a particular MHC/peptide complex are then detected and isolated using the methods described above. The cells, preferably after several rounds of proliferation to increase their numbers, are then administered (e.g., intravenously, intraperitoneally) to the subject to confer adoptive immunity. Such a procedure may be of particular utility in stimulating adoptive immunity against weak antigens such as tumor-associated antigens.

In another aspect, the present invention provides methods for stimulating or activating T cells, in vivo or in vitro. As noted above, the present invention provides for the production of soluble Class II MHC fusion proteins for which no soluble counterparts had previously existed, and for the production of multivalent and multimeric Class I and Class II MHC binding domains at higher valencies than previously obtained. Thus, these monovalent, multivalent and multimeric MHC binding domain fusion proteins and conjugates, loaded with appropriate MHC binding peptides and defining a specific MHC/peptide complex, may now be contacted with T cells in solution, in vivo or in vitro, to specifically stimulate or activate T cells which are reactive to the defined MHC/peptide complex. As noted above, the multivalent and multimeric MHC binding domain fusion proteins and conjugates of the invention are expected to be particularly potent for these purposes. When conducted in vivo (e.g., when the MHC binding domain fusion proteins or conjugates of the invention are administered as a pharmaceutical preparation), this serves as a method of vaccination against the MHC binding peptide when presented in the defined MHC/peptide complex. When used for vaccination purposes against pathogens including the MHC binding peptide, of course, the MHC binding domain components of the fusion proteins or conjugates are chosen to syngeneic to the subject being vaccinated.

In another aspect, the monovalent, multivalent or multimeric MHC binding domain fusion proteins and conjugates of the present invention may be used to kill or anergize T cells reactive to a defined MHC/peptide complex, or to tolerize an individual to a particular MHC/peptide complex. For example, the MHC binding domain fusion proteins may include Fc regions which activate the complement system and, thereby, cause the destruction of T cells to which they bind. Alternatively, the fusion proteins may be designed to include a cytotoxic substance attached to, for example, the C-terminus, or at some other point which does not interfere with the binding of the MHC/peptide complex to cognate T cell receptors (e.g., to a dimerization domain, Fc domain, ligand tag domain, or flexible molecular linker). Similarly, the MHC binding domain conjugates may be designed to include a cytotoxic substance attached to the MHC binding domains, to fusion protein components (e.g., a dimerization domain, Fc domain, ligand tag domain), to flexible molecular linkers or conjugating moieties, or to the carrier. For MHC binding domain conjugates in which the carrier is hollow (e.g., a liposome or hollow bead) orporous (e.g., a dendrimer or porous bead), a cytotoxic substance may be included within the interior or pores of the carrier. For these embodiments, useful cytotoxic substances include, for example, genistein, ricin, diphtheria toxins, Pseudomonas toxins, and radioactive isotopes (e.g., ¹²⁵I). It is also known in the art that high doses of many antigens have a T cell tolerizing or anergizing effect rather than a T cell stimulating effect. Therefore, administration of high doses of a monovalent, multivalent or multimeric MHC binding domain fusion protein or conjugate of the invention can cause tolerization to the MHC/peptide complex, even when lower doses would cause sensitization (i.e., vaccination or immunization). In cases where the goal is to tolerize an individual to an antigen which is normally presented by the subject's own MHC molecules, the MHC binding domain components of the fusion protein or conjugate are chosen so as to be syngeneic with the subject. Such cases would include tolerization to the antigens which cause allergic reactions, as well as autoantigens which are implicated in autoimmune disease. In other cases, however, the MHC components may be specifically allogeneic so as to tolerize the subject to an MHC/peptide complex which is foreign. Such cases would include tolerization to foreign tissue before or after organ or tissue transplantation in which the donor and recipient are not identical with respect to one or more MHC alleles.

By the term “effective amount,” with respect to tolerizing an individual to an MHC/peptide complex, is meant an amount sufficient to render T cells, otherwise specific for the MHC/peptide complex, unresponsive to the MHC/peptide complex. T cells which are unresponsive fail to activate or proliferate when presented with the complex for which they are specific. By the term “effective amount,” with respect to immunizing an individual to an antigen, is meant an amount sufficient to induce an immune response which results in activation or proliferation of T cells specific for the antigen in an MHC/peptide complex. Typical ranges of dosages are from 1 nanogram/kilogram to 100 milligrams/kilogram or even 500 milligrams/kilogram of body weight. Effective amounts will vary according to such factors as age, sex and sensitivity to the antigen.

Particular alleles of the MHC have been associated with a variety of diseases, including multiple sclerosis (MS), rheumatoid arthritis (RA), pemphigus vulgaris(PV) and systemic lupus erythematosus (SLE), and it has been postulated that these diseases are, at least in part, autoimmune in nature. That it, is has been suggested that particular MHC proteins “improperly” recognize processed self peptides presented to T cells in the form of complexes with MHC Class I or Class II molecules. In order to demonstrate this, MHC binding domain fusion proteins or conjugates which can be loaded with a single self-peptide implicated in the disease can be used. For example, using such MHC/peptide complexes, in the form of fusion proteins or conjugates, one can probe lesions in MS patients to determine whether the infiltrating T cells are reactive against a particular self peptide bound to a particular syngeneic MHC molecule. More generally, the MHC binding domain fusion proteins and conjugates of the invention may be used to detect T cells having any defined specificity by constructing an MHC binding domain fusion protein or conjugate loaded with the appropriate MHC binding peptide (covalently or noncovalently joined) and detecting the binding and/or activation of T cells contacted with the MHC binding domains.

Thus, as an example only, consider the diagnostic and therapeutic utilities of the present invention with respect to MS. The contribution of the MHC to MS susceptibility has been examined in a large number of studies (reviewed in Spielman and Nathenson, 1982; Hillert et al., 1994). These studies demonstrated that susceptibility is associated with the MHC class II region and that particular MHC class II haplotypes confer an increased risk. The strongest association is with the BLA-DR2 haplotype (DRB1*1501); approximately 50 to 60% of MS patients and 20% of normal subjects carry this haplotype. The DR2 haplotype is most common in MS patients from Western Europe, the U.S. and Canada; the haplotype is also increased among MS patients worldwide. Other MHC class II haplotypes (DR4, DR6) have been associated with susceptibility to MS in particular populations (Italians, Jordanian Arabs); however, these associations are not as strong as the association with DR2 (Marrosu et al., 1988; Kurdi et al., 1977).

HLA-DR2 (encoded by the DRA, DRB1*1501 genes) has been shown to present at least two peptides of human myelin basic protein (residues 85-99 and 148-162) to T cells. The MBP(85-99) peptide binds with high affinity to purified DR2, and the affinity of the MBP(148-162) peptide is lower but significant. DR2 transfectants (DRA, DRB1*1501) were found to present these MBP peptides to T cell clones thathad been generated fromblood lymphocytes of MS patients (Chou et al., 1989; Pette et al., 1990, Martinet al., 1990; Ota et al., 1990; Wucherpfennig et al., 1990; Valli et al., 0.1993, Wucherpfennig et al., 1994). These studies support the hypothesis that T cells specific for MBP and other myelin antigens are involved in the inflammatory response in MS. Direct identification of such T cells in MS lesions is, however, required to prove this hypothesis and this will require soluble, stable MHC complexes with single peptides, such as those provided by the present invention. In addition, therapeutic intervention, whether by tolerization or killing of T cells, will require soluble, stable MHC complexes with a high avidity for binding T cells specific for particular MIC/peptide complexes, such as the multivalent and multimeric MHC binding domain fusion proteins and conjugates provided by the present invention.

A principal difficulty with using soluble MHC/peptide complexes as probes and therapeutics is that the affinity for the TCR is relatively low. T cells compensate for the relatively low affinity of TCRs for MHC/peptide complexes by the interaction of multiple TCR molecules with MHC/peptide complexes on the surface of antigen presenting cells. Indeed, such dimerization of MHC Class II molecules may be important in T cell activation since HLA-DR1 is found as a dimer when crystallized (Brown et al., 1993). The present invention, by providing multivalent and multimeric MHC fusion proteins and conjugates addresses this problem. Classical studies with antibodies and their F(ab) fragments have demonstrated that bivalent/multivalent binding results in a striking increase in the ‘functional affinity’ (also termed the ‘avidity’). IgG molecules and F(ab) fragments bind monovalent antigen in solution with equal affinity; however, the binding to multivalent antigens (i.e. cell surface antigens) is greatly strengthened by the bivalent nature of the IgG molecule (Crothers and Metzger, 1972; Dower et al., 1984; Hornick and Karush, 1972). Indeed, the ‘functional affinity’ of IgG antibodies was found to be approximately 100-fold greater for bivalent than for monovalent binding, and this enhancement factor was even greater for multivalent binding by IgM antibodies (factor of 10³ to 104). Thus, the multivalent and multimeric MHC binding domain fusion proteins and conjugates of the present invention are expected to have far greater avidity for their cognate TCRs than standard, solubilized MHC proteins.

### EXAMPLES

A. Monomeric MHC Binding Domain-Coiled Coil Dimerization Domain Fusion Proteins

1. DNA Constructs for Monomeric MHC Binding Domain Fusion Proteins. The extracellular domains of the HLA-DR2 α chain (residues 1-191 of DRA*0101) and β chain (residues 1-198 of DRB1*1501) were expressed as fusions withthe 40 amino acid leucine zipper dimerization domains of Fos or Jun, respectively (van Straaten et al., 1983; Angel et al., 1988). The entire extracellular domains, rather than C-terminally truncated domains, were employed because charge-charge interactions between the DRcc Glu at position 191 and the DRY Lys at position 198 are thought to facilitate assembly (Cosson and Bonifacino, 1992) of these molecules. The extracellular domains of DRcc and DRY as well as the Fos and Jun dimerization domains were generated by PCR with primers designed to include a seven amino acid linker (VDGGGGG, residues 199-205 of SEQ ID NO: 2) with a SalI restriction site at the C-terminus of the MHC extracellular domains and at the N-terminus of the Fos or Jun leucine zipper domains. The MHC segments were then joined with the Fos or Jun segments through the SalI restriction site. This linker was included between the DR and leucine zipper segments both to facilitate cloning (through the SalI site) and to allow for greater rotational freedom of the chains (through the poly-Gly sequence). These constructs were reamplified by PCR to permit cloning into the XhoI-EcORI sites of pPIC9 as in frame fusions with the α-mating factor secretion signal. The in-frame cloning into this vector preserved the Lys-Arg-Glu recognition sequence (cleavage C-terminal to Arg) required for cleavage of the α-mating secretion signal by the KEX2 gene product (Brake, 1990).

The following oligonucleotides were used for the construction: DRa forward primer 5′ GTA TCT CTC GAG AAA AGA GAG ATC AAA GAA GAA CAT GTG ATC 3′, XhoI site underlined (SEQ ID NO: 5); DRa reverse primer 5′ GTC ATA GAA TTC TCA ATG GGC GGC CAG GAT GAA CTC CAG 3′, EcORI site underlined (encodes 3′ end of Fos segment, stop codon and EcORI restriction site) (SEQ ID NO: 6); DRβ forward primer 5′ GTA TCT CTC GAG AAA AGA GAG GGG GAC ACC CGA CCA CGT TTC 3′, XhoI site underlined (SEQ ID NO: 7); DRβ reverse primer 5′ GTC ATA GAA TTC TCA ATG GTT CAT GAC TTT CTG TTT AAG 3′ EcORI site underlined (encodes 3′ end of Jun segment, stop codon and EcORI restriction site) (SEQ ID NO: 8). The resulting PCR products are disclosed as SEQ ID NO: 1 and SEQ ID NO: 3. These PCR products were cloned into the XhoI-EcORI sites of pPIC9 and were verified by restriction mapping and dideoxy-sequencing.

These constructs were first tested in CHO cells (using the native DR α and β chain signal peptides). CHO transfectants were found to assemble and secrete DR αβ heterodimers indicating that the Fos/Jun leucine zipper promoted the proper assembly of DR2 molecules (data not shown). As described below, however, higher levels of expression were obtained in a yeast expression system employing Pichia pastoris. Recent work has demonstrated that Drosophila Schneider cells give the highest level of protein production (˜1 mg/liter, compared to 0.3 mg/liter in Pichia pastoris).

2. Transformation of Pichia with MHC Binding Domain Fusion Protein Constructs. For protein production, the DRα-Fos and DRβ-Jun constructs were expressed in Pichia pastoris under the control of the alcohol oxidase (AOX1) promoter. Pichia pastoris was chosen because stable transformants can be rapidly generated and screened; in addition, several secreted proteins have been produced at very high levels in this system (Cregg et al., 1993).

To direct expression to the secretory pathway, DRα and β chains were cloned into Pichia pastoris expression vector pPIC9 as in frame fusions with the α-mating factor secretion signal (Brake, 1990). The α-mating factor secretion signal is cleaved by the KEX2 gene product at the sequence Leu-Glu-Lys-Arg-Glu (residues 3-7 of SEQ ID NO: 2 and SEQ ID NO: 4), with the cleavage C-terminal to the Arg residue. Although this design results in the addition of a glutamic acid residue to the N-termiinus of the mature DRα and DRβ chains, the N-termini of these chains are located in a manner that this additional residue should not affect the assembly of the heterodimer. Molecules expressed as fusions with the α-mating factor secretion signal were efficiently secreted while usage of the PHO1 secretion signal (vector pHL-S1, Invitrogen, San Diego, Calif.) resulted in little or no secretion. For transformation, the expression cassette of pPIC9 can be excised as a BglII fragment; the cassette carries 5′ and 3′ sequences of the AOX1 gene to allow for integration into the AOX1 locus as well as the HIS4 gene that allows for selection of transformants inhistidine deficient media. Genes integrate into the AOX1 locus by homologous recombination; integration into the AOX1 gene disrupts the gene and leads to slow growth if methanol is the only carbon source (methanol utilization deficient phenotype, Mut^{s}) (Cregg et al., 1987).

Thus, pPIC9 plasmid DNA was purified on CsCl gradients and digested with BglII to release the expression cassette (5′ end of AOX1 gene-DRγ or DRβ chain construct-polyadenylation signal-HIS4 gene-3′ end of the AOX1 gene). Transformations were done by spheroplasting of the GS115 strain (following the procedure provided by Invitrogen, San Diego, Calif.). Briefly, GS115 cells were grown to mid-log phase in YPD media and spheroplasts were prepared by limited digestion of the yeast cell wall with zymolase (approximately 70% of spheroplasting) (Cregg et al., 1987). Cells were transfected with 5 mg of DRα and DRβ plasmid DNA and transfectants that expressed the HIS4 gene (present in the pPIC9 expression cassette) were selected on HIS⁻ plates. Integration of plasmids into the AOX1 locus was confirmed by replica plating of colonies on minimal media plates with methanol or dextrose as the sole carbon source. Transformants that had integrated the plasmid DNAinto the AOX1 locus showed little or no growth on methanol plates due to disruption of the alcohol oxidase gene.

3. Identification of Recombinant Colonies. A major advantage of the Pichia pastoris system is that transformants can be readily identified: Integration into the AOX1 locus confers a methanol utilization deficient (Mut^{s}) phenotype that can be determined by comparing the growth of duplicate colonies on plates with methanol or dextrose as the sole carbon source. Mut^{s} colonies obtained after cotransformation of plasmids carrying the DRα and DRβ chain constructs were tested by PCR analysis of genomic DNA for the integration of DRα and β chain genes. 27 of 28 colonies with aMuts phenotype carried DRax and/or DRO chain genes; four of these colonies (14.2%) had integrated both genes.

Thus, briefly, integration of DRo and DRβ chain constructs was examined by PCR analysis of genomic DNA isolated from individual Muts colonies. Replica colonies were transferred into 200 ml of lysis buffer (2.5 M LiCl, 50 mM Tris, pH 8.0, 4% triton X-100, 62 mM EDTA) using a sterile toothpick. Acid washed glass beads and an equal volume of phenol/chloroform (1:1) were added and samples were vigorously vortexed. Following centrifugation, the upper phase was transferred to a clean tube and genomic DNA was precipitated by addition of 2.5 vol of cold EtOH. Following incubation at −20° C. for 20 minutes, the pellet was collected by centrifugation, washed with cold 70% EtOH and air-dried. DNA was resuspended in 40 ml of sterile water and denatured at 94° C. for 10 minutes; 10 ml of DNA was used for each PCR reaction. DRα and DRβ chains were amplified by PCR for 35 cycles (94° C. 1 min, 55° C. 2 min, 72° C. 2 min) using the oligonucleotides that had been used to generate the DNA constructs; PCR products were resolved on 1% agarose gels stained with ethidium bromide.

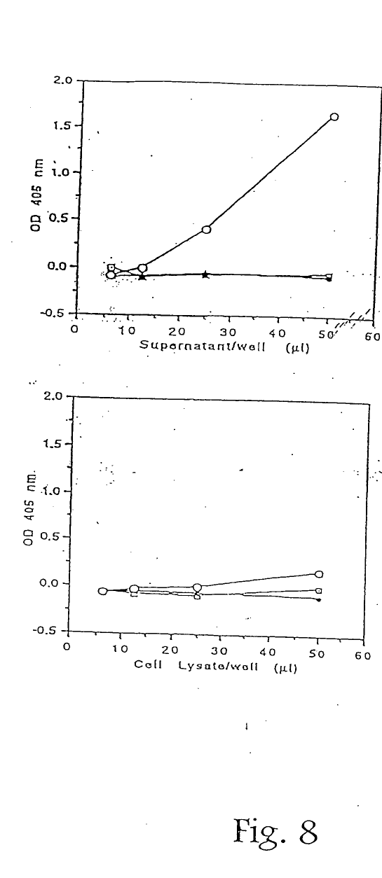

4. Expression and Purification of Monomeric MHC Binding Domain Fusion Proteins. The four transformants that carried both DRα and β chain genes were examined for the expression of DR2 binding domain heterodimers. Cells were grown for two days in media containing glycerol as the sole carbon source and were then switched to media containing 0.5% methanol. Supernatants and cell lysates were examined by sandwich ELISA using a mAb specific for the DR coo heterodimer (mAb L243) for capture and a polyclonal DR antiserum (CHAMP) for detection. DR αβ heterodimer was detected in the cell lysates and supernatants of DR αβ transfectants. Transformants that carried only DRα or DRβ chain genes were used as controls; cell lysates and supernatants from these cells were negative in the assay (FIG. 8). These experiments demonstrated that the DR αβ binding domain heterodimer was assembled and efficiently secreted. The four Pichia clones showed similar expression levels; this is not surprising because all four transformants had integrated the genes into the AOX1 locus.

For large scale expression, cells were grown in a high density fermenter and DR2 MHC binding domain fusion proteins were purified from concentrated supernatants by affinity chromatography with the L243 mAb. The mAb used for purification (L243) binds to the DRα chain but only when properly assembled with the DRβ chain. Affinity purification yielded approximately 300-400 mg of HLA-DR2 fusion protein per liter of culture. SDS-PAGE revealed two bands, the identity of these bands (upper band DRα, lower band DRβ) and appropriate cleavage of the α-mating factor signal peptide were confirmed by N-terminal sequence analysis following separation of DRα and β chains by SDS-PAGE and transfer to a PVDF membrane.

HPLC gel filtration analysis (Bio-Gel SEC 300 mm×7.8 mm; flow rate 1 ml/min, PBS pH 6.8) of 10 μg of the HLA-DR2 fusion protein demonstrated that the recombinant fusion protein eluted as a single symmetric peak and only very small amounts of higher molecular weight aggregates were detected. In contrast, HLA-DR1 expressed in a Baculovirus system was found to aggregate unless these molecules were loaded with a high affinity peptide (Stern and Wiley, 1992). These data demonstrated that the DR2 αβ heterodimer was assembled and secreted in the Pichia pastoris expression system even in the absence of a high affinity peptide. Importantly, the purified molecules did not aggregate even though they had not been loaded with a high affinity peptide.

Induction of high density cultures was carried out using a Inceltech LH series fermenter equipped with monitors and controls for pH, dissolved O₂, agitation, temperature, and air flow. A 100 ml YNB-glycerol overnight culture was used to inoculate the fermenter which contained 10 liters of fermentation basal salts medium (0.93 g/L calcium sulfate 2H₂O, 18.2 g/L potassium sulfate, 14.9 g/L magnesium sulfate 7H₂O, and 6.5 g/L potassium hydroxide) containing 4% glycerol (w/v) plus 43.5 ml of PTM₁ trace salts (24 mM CuSO₄, 0.53 mM NaI, 19.87 mM MnSO₄, 0.83 mM Na₂MoO₄, 0.32 mM boric acid, 2.1 mM CoCl₂, 0.15 mM ZnCl₂, 0.23 mM FeSO₄, and 0.82 mM biotin) at 30° C. Dissolved O₂ was maintained above 20% by adjusting aeration and agitation, and pH was maintained at 6.0 by the addition of 28% (v/v) ammonium hydroxide. Growth was continued until the glycerol was exhausted (20 hours). A glycerol fed-batch phase was initiated by the limited addition of 50% (w/v) glycerol and 12 ml PTM₁ salts per liter of glycerol at 18.15 ml/hr/L initial fermentation volume until the culture reached a wet cell weight (wcw) of 200 g/L (22 hours). After the glycerol fed-batch phase, the culture was induced by replacing the glycerol feed with a methanol-batch feed (100% methanol containing 12 ml PTM₁ trace salts per liter of methanol) at 1 ml/hr/L. The methanol feed was gradually increased in 10% increments every 30 minutes to a rate of 3 mlhr/L and the fermentation continued for a duration of 96 hours.

Supernatants were concentrated by ultrafiltration on a YM30 membrane (Amicon) and passed over an anti-DR (mAb L243) affinity column at aflow rate of approximately 10 ml/hour. Following extensive washing with PBS, heterodimers were eluted with 50 mM glycine, pH 11.5. Eluates were immediately neutralized by addition of 2 M Tris, pH 8.0, dialyzed against PBS and concentrated by ultrafiltration. Protein concentrations were determined by Coomassie Plus Protein Assay (Pierce, Rockford, Ill.) using bovine serum albumin as a standard.

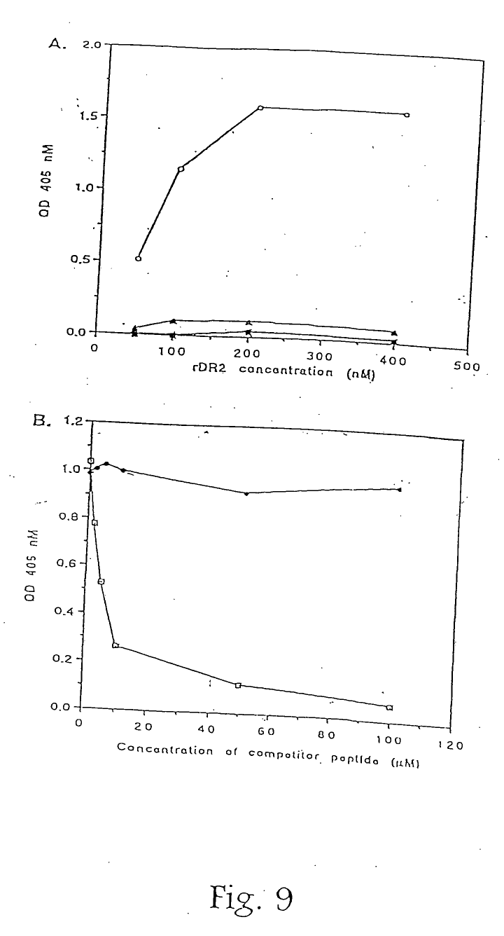

5. Peptide Loading of MHC Binding Domain Fusion Proteins. A human myelin basic protein fragment (residues 85-99) that is recognized by DR2 restricted T cell clones from MS patients was previously shown to bind with high affinity (IC50 of 4.2 nM) to detergent soluble DR2 purified from L cell transfectants (Wucherpfennig et al., 1994 and 1995a). A biotinylated peptide with an SGSG linker between the biotin moiety and the MBP sequence (i.e., biotin-SGSG-MBP(85-99)) was used to examine the specificity of peptide binding to the recombinant DR2 fusion proteins. Peptide binding was assessed by incubating DR2 fusion proteins (50-400 nM) with the biotinylated peptide (2 μM) at 37° C. for different periods of time; non-biotinylated peptide was used as a competitor to demonstrate the specificity of binding (FIG. 9). DR2/peptide complexes were then captured on an ELISA plate using the L243′ mAb, and the amount of bound biotinylated peptide was quantitated using peroxidase-labeled streptavidin and ABTS as a peroxidase substrate (detection at 405 nm).

Peptide binding to the DR2 fusion proteins was strongly dependent on the pH, with a maximum observed at pH 7 to pH 8; relatively little binding was observed at pH 5. A similar pH optimum had previously been observed for binding of the MBP peptide to detergent soluble DR2 (Wucherpfennig et al., 1994). Binding of peptide was dependent on the relative molar ratio of DR versus peptide, with a maximum of binding at a 10-fold molar excess of peptide over DR2 FIG. 9). Binding was shown to be specific because it could be blocked by an excess of non-biotinylated MBP(85-99) peptide, but not by an analog peptide in which the P1 anchor residue (Val 89) of MBP(85-99) had been substituted by aspartic acid (FIG. 9).

To determine what fraction of the MHC binding domain fusion proteins could be loaded with a single peptide, complexes of the DR2 fusion proteins and the biotinylated MBP peptide were precipitated with streptavidin beads. Following precipitation, DRx and β chains were resolved by SDS-PAGE and detected by Western blotting using a polyclonal DR antiserum. Approximately 50% of the molecules were precipitated with streptavidin beads and 50% remained in the supernatant. Control experiments demonstrated that precipitation of the DR2/peptide complexes was specific as the molecules were not precipitated when control agarose beads, an unlabeled MBP peptide or an excess of unlabeled peptide over biotinylated peptide were used; rather, the DR2 fusion proteins remained in the unbound fraction.

For immunoprecipitation experiments, the DR2 fusion protein (400 nM) was incubated with biotinylated peptide (2 μM) in a 50 ml volume in PBS, 1 mM EDTA, 1 mM PMSF, pH 7.2 for 24 hours at 37° C. DR2-peptide complexes were precipitated with streptavidin-agarose beads. Beads were first blocked with 3% bovine serum albumin in PBS, 0.1% NP40 for 1 hour at 4° C.; beads were then pelleted and the DR2/peptide samples added. Following a 1 hour incubation, beads were washed three times with blocking buffer. DR2-peptide complexes were eluted from streptavidin beads by heating in 1×SDS-PAGE buffer at 94° C. for 3 minutes. Samples were resolved on a 12.5% SDS-PAGE and transferred to immobilon membrane (Millipore). Blots were blocked overnight with 5% non-fat dry milk in 50 mM Tris, pH 8.0, 150 mM NaCl, 0.2% Tween 20 (TBST buffer). Precipitated DR α and β chain fusions were detected with a polyclonal DR antiserum (CHANP, 1:50,000 in blocking buffer for 90 min). Blots were washed in TBST buffer and incubated for 30 min with a peroxidase conjugated anti-rabbit IgG antibody (1:10,000 in blocking buffer). Following extensive washing in TBST, bands were detected by enhanced chemiluminescence (Amersham, Arlington Heights, Ill.).

In a separate set of experiments, peptide binding to recombinant DR2 fusion proteins was quantitated by capturing DR2 fusions to ELISA plates with an immobilized DR antibody. Standard binding conditions were: 37° C. for 24 hours in PBS, pH 7.2, 1 mM EDTA, 1 mM PMSF. Following peptide binding, bound peptide was quantitated by ELISA. Plates were coated with 200 ng/well of the purified L234 mAb in 0.1 M bicarbonate, pH 9.6 overnight at 4° C. Non-specific binding sites were blocked with 3% BSA in PBS, 0.05% Tween 20 for 2 hours. Samples were diluted in blocking buffer and added to the wells (1 hour). HLA-DR2 bound biotinylated peptide was quantitated with streptavidin-peroxidase using ABTS as a peroxidase substrate; absorbance was read at 405 nm.

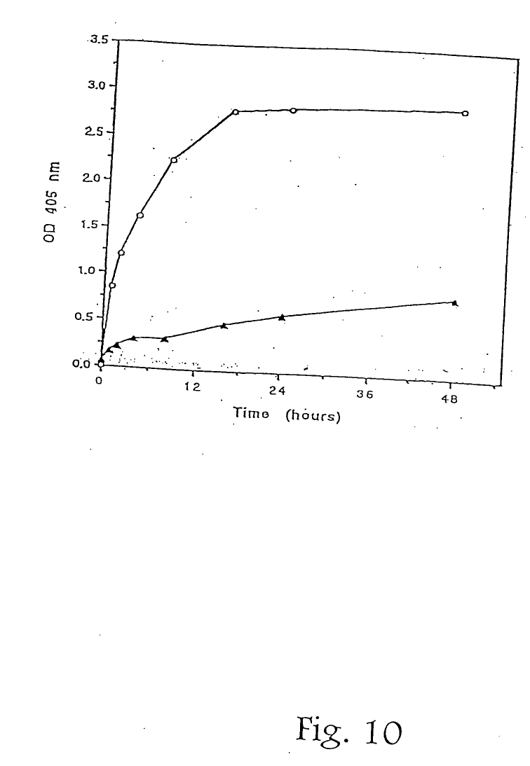

6. Kinetics of Peptide Binding to MHC Binding Domain Fusion Proteins. The kinetics of peptide binding by detergent soluble DR2 purified from an EBV transformed B cell line (Gorga et al., 1987) and by recombinant DR2 MHC binding domain fusion proteins were compared (FIG. 10). Equimolar amounts of both DR2 preparations (200 nM) were incubated with the biotinylated MBP peptide (2 μM) at 37° C. for different periods of time; the amount of DR-bound peptide was examined by ELISA using the DR specific L243 mAb for capture and streptavidin-peroxidase for detection of DR-bound peptide. The kinetics of peptide binding were strikingly different: With the recombinant MHC binding domain fusion proteins of the invention, the kinetics of binding were much faster and a much larger fraction of the molecules were loaded (50% maximum binding after only 3 hours with a plateau after 18 hours). In contrast, the kinetics of peptide binding to DR2 from B cells were slow; the fraction of peptide loaded molecules slowly increased over a 48 hour period without reaching a plateau (FIG. 10). These results may be explained by the fact that the majority of DR molecules purified from B cells are already occupied with high affinity peptides, as demonstrated by peptide elution studies and crystallization of HLA-DR1 (Chicz et al., 1993; Brown et al., 1993). In contrast, the peptide binding site of a large fraction of the recombinant DR2 fusion proteins is empty and readily available for binding by a high affinity peptide.

7. Production of HLA-DQ MHC Binding Domain Fusion Proteins. The leucine zipper dimerization domains of Fos and Jun were also used to express soluble HLA-DQ MHC binding domain fusion proteins for DQ1 and DQ8 alleles, which are associated with susceptibility to pemphigus vulgaris and insulin dependent diabetes, respectively. The same design was used as described above for recombinant DR2 (including splice points). Stable transfectants were generated using Drosophila Schneider cells and soluble DQ molecules were affinity purified. Peptide binding studies using peptides that were previously shown to bind to DQ 1 or DQ8 demonstrated that the molecules were functional.

B. Divalent MHC Binding Domain-Immunoglobulin Fusion Proteins

1. DNA Constructs for Divalent MHC Binding Domain Fusion Proteins. Divalent HLA-DR2 MHC binding domain fusion proteins were expressed by fusing the Fc part of IgG2a to the 3′ end of the DRa-Fos cDNA construct described above. In this design, the DRo-Fc chain corresponds to an antibody heavy chain and the DRO-Jun construct to an antibody light chain. The DR2-IgG design was chosen both to increase the affinity for the T cell receptor by increasing valency, and to attach an effector domain, the Fc region of IgG2a. Complement fixation may result in the lysis of target T cellsfollowing binding of DR2-IgG molecules to the T cell receptor. DR2-IgG molecules may therefore be useful for the selective depletionof autoaggressive T cells.

The Fc part of IgG2a was amplified by RT-PCR from a mouse hybridoma (L243) that secretes an IgG2a mAb. The PCR product was fused in frame with the DRa-Fos construct by overlapping PCR with a primer for the Fc part that overlapped by 20 bp with the 3′ end of the DRx-Fos construct. DRa-Fos and Fc were amplified separately, gel purified, mixed and amplified using oligos representing the 5′ end of DRa and the 3′ end of IgG2a. The construct was cloned into the EcORI-BamHI sites of the pRmHa-3 expression vector under the control of the metalothionein promoter. The insert was checked by restriction mapping and dideoxy-sequencing.

2. Expression of Divalent MHC Binding Domain Fusion Proteins. DR2-IgG fusion proteins were expressed in the Drosophila Schneider cell system. The Drosophila Schneider cell system was chosen for the expression of the DR2-IgG fusion protein for the following reasons: (1) recombinant antibodies have previously been expressed in insect cells, (2) in the pRmHa-3 expression vector, genes are under the control of the strongly inducible metalothionein promoter, (3) Schneider cells can be grown to a high cell density in serum free media, and (4) large scale production of protein is more straightforward than in another insect cell system (the Baculovirus system) since stable transfectants are generated.

Stable transfectants were generated by the contransfecting Schneider cells with the DRα-IgG and DRβ chains vectors as well as with plasmid pH8CO. This vector confers resistance to selection by methotrexate. Transfectants were selected with 0.1 μM methotrexate in Schneider media, 10% fetal calf serum. Transfectants were cloned by limiting dilution, and the secretion of DR2-IgG fusion proteins was examined by ELISA using an antibody specific for the Fc segment of IgG, as well as an antibody specific for the DRαβ heterodimer.

Transfectants were grow to a density of ˜10×10⁶/ml and expression was induced by adding CuSO₄ to a final concentration of 1 mM. Supernatants were harvested five days following induction and concentrated by ultrafiltration. DR2-IgG fusion proteins were purified by affinity chromatography using the L243 mAb. Purity was examined by SDS-PAGE; for comparison, purified mouse IgG was also run on the gel. Western blot analysis with a polyclonal antiserum confirmed the identity of the two bands. Peptide binding experiments demonstrated that DR2-IgG fusion proteins were properly folded and functional.

C. Decavalent MHC Binding Domain-Immunoglobulin Fusion Proteins

DR2-IgM fusion proteins molecules comprise ten MHC binding domains (five IgM monomers per IgM pentamer; two MHC binding domains per IgM monomer). Since DR2-IgG fusion proteins have only two MHC binding domains, the functional affinity of DR2-IgM fusion proteins for cognate T cell receptors is expected to be much higher. A significant increase in affinity would improve the sensitivity for immunohistochemical staining as well as the therapeutic effectiveness of these molecules. DR2-IgM fusion proteins may be particularly useful for immunotherapy for the following reasons: (1) higher avidity for the T cell receptors on cognate T cells, (2) complement fixation by the Fc segment of IgM, and (3) longer serum half life.

The Fc segment of IgM is fused in frame to the 3′ end of the DRα-Fos segment, as previously described for the DRα-IgG construct. The DRα-IgM construct is cloned into, for example, the EcORI-BamHI sites of the pRmHa-3 expression vector, under the control of the inducible metalothionein promoter (Bunch et al., 1988). The DRα-IgM and DRβ chain fusion constructs are cotransfected with a gene encoding the J-chain. The J-chain facilitates assembly and secretion of IgM molecules by mammalian cells (Matsuuchi et al., 1986). The J-chain may be cloned into, for example, expression vector pUC-hygMT which confers resistance to hygromycin. Stable transfectants may then be selected using hygromycin at 100 μg/ml in Schneider cell media (Sigma) supplemented with 10% insect cell tested fetal calf serum. Transfectants are cloned by limiting dilution and tested for expression of DR2-IgM fusion proteins following induction with CuSO₄.

Secretion of DR2-IgM fusion proteins may be assessed by immunoprecipitation with mAb L243, followed by Western blot analysis with antibodies specific for the Fc segment of the IgM. For protein production, transfectants can be adapted to serum free media (ExCell 400, JRH Biosciences).

These constructs also can be transfected into CHO cells or into a murine B cell line (M12.C3). CHO cells were previously shown to secrete recombinant IgM antibodies at high levels and have been used for the expression of a CD2-IgM fusion protein (Wood et al., 1990; Arulanandam et al., 1993). For expression in these cells lines, the DRct-IgM and DRO chain constructs are cloned into eukaryotic expression vectors. The DRx-IgM construct can be cloned into, for example, pcDNA3, which carries the neomycin resistance gene, and the DRO-Jun construct can be cloned in the pcDNAI vector (Invitrogen, San Diego, Calif.). Cells can be transfected by electroporation and stable transfectants can be selected with G418. Secretion of DR2-IgM fusion proteins can be assessed by immunoprecipitation with mAb L243 and by Western blot analysis.

In initial experiments, DR2-IgM fusion proteins were not secreted by Drosophila Schneider cells and, therefore, expression in COS cells was performed. DR2-IgM fusion proteins were secreted when COS cellswere transfected with the cDNA constructs.

D. Multivalent MHC Binding Domain-Ligand-Tag Fusion Proteins

1. DNA Constructs for MHC Binding Domain-Biotin-Tag Fusion Proteins. Biotin ligase specifically biotinylates a lysine residue within a 0.14-amino acid recognition sequence (LGGIFEAMKLRD, SEQ ID NO: 9) (Shatz, 1993) and, therefore, a DNA sequence encoding this sequence was added to the DRα-Fos construct. This “DRa-Fos-tag” construct was cloned into the EcORI and SalI sites of Drosophila expression vector pRmHa-3 under the control of the inducible metalothionein promoter. Drosophila Schneider cells stably co-transfected with the DRα-Fos-tag and DRβ-Jun constructs were generated as described above for the DR2-IgG fusion proteins. The resulting “DR2-tag” fusion molecules differ from the DR2-Fos/Jun fusion proteins only by the addition of the biotinylation sequence tag to the C-terminus of the DRα-Fos construct. The DR2-tag fusion proteins were affinity purified from supernatants using the L243 mAb as described above.

Site specific biotinylation of these DR2-tag molecules allows assembly of DR2-tag-biotin tetramers on avidin or streptavidin because avidin and streptavidin have four biotin binding sites. Thus, tetramers are made by mixing the DR2-tag-biotin molecules and streptavidin at a 4:1 molar ratio.

2. Biotinylation of MHC Binding Domain-Biotin-Tag Fusion Proteins. A biotin ligase cDNA (provided by S. Lesley, Promega Corporation) was cloned as a NdelI-XhoI fragment into the prokaryotic expression vector pET22b under the control of the T7 promoter. This construct was transfected into E. coli strain BL21/DE3 which islysogenic for the T7 RNA polymerase gene under the control of the lacZ promoter. Protein expression was induced by addition of IPTG to 1 mM for 4 hours. Cells were then harvested by centrifugation, resuspended in 20 mM Tris, pH 8.0, 100 mM NaCl. Cells were sonicated and insoluble material was removed by centrifugation, yielding 5 ml of a soluble cytoplasmic protein fraction from 100 ml of culture.

Biotinylation was performed at 37° C. in a 100 μl volume with 0.1 to 100 μl of enzyme, 1 mM of ATP and 1 or 10 μM of biotin. Following the reaction, recombinant DR2-tag-biotin molecules were captured on a 96-well plate coated with the L243 mAb and the degree of biotinylation was quantitated using peroxidase conjugated streptavidin. A Western blot was sequentially probed with a polyclonal DR antiserum and with streptavidin peroxidase. This experiment demonstrated specific biotinylation of the DRat chain (which carried the 14-amino acid biotin ligase recognition sequence) by biotin ligase.

Fluorescein-labeled streptavidin was used to examine the formation of DR2-tag-biotin tetramers. Fluorescein absorbs at 492 nm, allowing detection during HLPC gel filtration chromatography (Bio-Gel SEC 300 mm×7.8 mm; flow rate 1 ml/min, PBS pH 6.8). Streptavidin (MW 60 kDa) eluted as a single peak at 8.3 minutes on the HPLC gel filtration column. The streptavidin-DR2-tag-biotin complex eluted at 5.8 minutes. Intermediates with one, two or three DR2 fusion molecules bound to streptavidin were observed when smaller amounts of DR2-tag-biotin were used for complex formation. MW standards confirmed the predicted molecular weight of streptavidin and the streptavidin-DR complex.

3. Peptide Loading of MHC Binding Domain Fusion Proteins. A (His)₆-tagged MBP(85-99) peptide was used to purify DR2 fusion proteins loaded with a single peptide by metal affinity chromatography. DR2-tag molecules were incubated with the (His)₆-tagged MBP peptide and precipitated with the metal affinity resin (Talon Metal Affinity Resin, Clontech). DR2/peptide complexes were eluted under mild conditions (1 mM EDTA). Eluted DR2 molecules were analyzed by Western blot analysis, using a polyclonal DR antiserum for detection These experiments demonstrated that defined DR2/peptide complexes can be generated at a yield of ˜50%.

4. Binding of T Cells to MHC Binding Domain-Biotin-Tag Fusion Proteins. The binding of T cell receptors on the surface of human T cell clones to tetravalent MHC binding domain biotin-tag fusion proteins was examined. Biotinylated DR2-tag molecules were loaded with the MBP(85-99) peptide and captured on a streptavidin coated plate; the binding of fluorescent-labeled T cells to immobilized DR2/peptide complexes was quantified. As a positive control, wells were coated with an anti-CD3 mAb.

Binding was examined using a human DR2 restricted T cell clone (Ob.1A12) specific for MBP(85-99) and a DR4 restricted control clone (Go.P3.1) specific for residues 190-204 of human desmoglein 3 protein. Specific binding to DR2 MBP(85-99) complexes was only observed with the MBP(85-99) specific T cell clone. Furthermore, binding was observed only with the DR2/MBP(85-99) complex, and not with empty DR2.

Binding was examined by capturing biotinylated DR2-tag molecules on a streptavidin coated plate. Non-specific binding sites were blocked with 0.1% BSA in PBS. T cells were labeled with BCEFC-AM, a fluorescent membrane probe, for 30 minutes at 37° C., washed and added to the plate for 20 minutes at 37° C. Following three washes, the fraction of T cells that bound to DR2/peptide complexes or to the anti-CD3 mAb was determined in a fluorescent plate reader.

E. MHC Binding Domain Conjugates with Bead Carriers

DR2-biotin tag molecules were used to generate highly multimeric MHC binding domain conjugates for the specific staining of antigen specific T cells. DR2/peptide complexes were bound to highly fluorescent microbeads, purchased from Molecular Probes (Eugene, Oreg.), to which streptavidin had been conjugated. Polystyrene beads similar in size to viral particles (40 nm) were selected based on their ability to remain soluble; these beads pellet in an ultracentrifuge but not under the low G-forces used to wash cells. Staining of antigen specific T cells was examined by FACS. For FACS staining, biotinylated mAbs specific for CD4 (positive control) and a murine MHC class II (10-2.16) (negative control) were used as controls. Approximately 10⁶ T cells were used for each assay. T cells were pelleted and resuspended in cold PBS, 0.1% sodium azide. Staining was observed with both DR2/MBP(85-99) specific T cell clones and multivalent DR2/MBP(85-99) peptide complexes; the staining intensity was similar to that observed with the CD4 mAb. Binding was highly specific because a single amino acid substitution in the MBP peptide at a TCR contact residue greatly reduce the staining intensity. No staining was observed for control T cell clones specific for other MHC class II/peptide combinations. These control clones were specific for MBP(85-99) bound to HLA-DQ1 (clone HY.1B11), a desmoglein 3 peptide (190-204) bound to HLA-DR4 (clone Go.P3) and a tetanus toxoid peptide (830-843) bound to BLA-DR2a (clone Kw-TTI).

F. MHC Binding Domains with Covalently Bound Peptides

DR2-Ig fusion proteins are generated to allow multivalent binding to TCRs on target T cells (2 DR2/peptide arms in the DR2-IgG fusion protein, 10 DR2/peptide arms in the DR2-IgM fusion protein). In order to ensure that all binding sites in these molecules will be loaded with the same peptide, DR2 molecules were expressed with a covalently linked MBP peptide. The MBP(85-99) sequence was attached to the N-terminus of the mature DR β chain through a 16-amino acid linker (linker sequence: SGGGSLVPRGSGGGGS, SEQ ID NO: 10). This cDNA construct was used to express DR2 molecules and DR2-IgG molecules with a linked MBP peptide in Drosophila Schneider cells.

G. Uses for MHC Binding Domain Fusion Proteins and Conjugates

1. Use of DR2-Ig Fusion Proteins for the Selective Depletion of T Cells. DR2-Ig fusions proteins may be useful for the selective depletion of T cells that recognize DR2 bound self-peptides. Binding of DR2-Ig fusion proteins by the T cell receptor may lead to complement fixation and lysis of target T cells. Multivalent DR2 molecules could also be conjugated to genistein, a tyrosine kinase inhibitor that induces apoptosis following uptake by target cells.

2. Affinity of Multivalent DR2/Peptide Complexes for the T Cell Receptor. The binding of multivalent DR2/peptide complexes to the TCR will be examined using human DR2 restricted T cell clones. DR2 molecules will be loaded with the MBP(85-99) peptide and labeled with [251] using immobilized chloramine T (Iodobeads, Pierce). In the binding assay, a fixed number of T cells (1×10⁶ cells, 1 ml) will be incubated with 6-10 different concentrations of radiolabeled DR2/peptide complexes in PBS, 1.0% BSA, 0.02% NaN₃. Radiolabeled molecules will be used at concentrations at which only a small fraction (less than 10%) of TCRs on target cells will be occupied.

First, the incubation time required to reach equilibrium will be determined; cell-bound and unbound DR2/peptide complexes will be separated by rapid (10-15 sec.) centrifugation through a layer of 84% silicone (d=1.050), 16% paraffin oil (d=0.838). Cell boundradioactivity will be quantitated in a y-counter and data will be analyzed on Scatchard plots to determine K (dissociation constant) and n (number of TCR molecules on target cells). Several of controls will be included to demonstrate specificity of binding: (1) T cell clones with an unrelated MHC/peptide specificity, (2) DR2/peptide complexes that were loaded with control peptides, and (3) Competition of binding by an excess of unlabeled DR2/peptide complexes.

It will be of particular interest to determine the kinetics of binding by monovalent (DR2), bivalent (DR2-IgG) and multivalent (DR2-IgM, DR2-tetramers) molecules to the TCR “On” rates will be determined by incubating target cells with radiolabeled ligands for different time periods at 37° C. (in the presence of 0.02% sodium azide to prevent internalization of the TCR), followed by rapid separation of reactants. “Off” rates will be determined by incubating T cells with labeled ligands until equilibrium is reached. Cells will then be washed, incubated for different periods of time and the amount of cell bound radioactivity will be determined. Off rates are expected to be significantly different for monovalent, bivalent and multivalent ligands. Classical studies with IgM antibodies have shown that multivalent attachment dramatically slows the dissociation of bound antibody (Crothers and Metzger, 1972; Hornick and Karush, 1972).

3. Complement-Mediated Lysis of T Cells Specific for DR2-Ig Fusion Proteins. The Fc segment of IgG2a was chosen for the DR2-IgG fusion protein because IgG2a fixes complement. IgM also fixes complement, allowing complement mediated lysis of target T cells by fusion proteins to be assessed. T cells will be incubated with DR2-IgG or DR2-IgM complexes; cells will then be washed and incubated with rabbit serum complement diluted in media (1:5 to 1:20 dilution). Rabbit serum complement will be obtained form Cedarlane Laboratories and will be pretested to ensure that the lot does not have nonspecific cytotoxicity against human T cells; complement will be aliquoted and stored at −70° C. Cytotoxicity will be determined after 30 and 60 minutes of incubation at 37° C. by trypan blue staining (% cytotoxicity [number of dead cells/number of live+dead cells]×100). A mAb specific for human CD3 (OKT3, IgG2a) that fixes complement will be used as a positive control. Specificity of lysis will be assessed using control T cell clones as well as DR2 molecules loaded with control peptides.

4. Coupling of DR2/Peptide Complexes to Toxins to Induce Apoptosis. Multivalent DR2 molecules of all three designs, DR2-IgG, DR2-IgM and DR2-tetramers, will be conjugated to toxin moieties as another means of mediating selective T cell death. Genistein, a tyrosine kinase inhibitor, may be particularly effective for this purpose. In a recent study, genistein coupled to CD19 mAb was found to be highly effective in eradicating a human B cell leukemia from SCID mice (Uckun et al., 1995). A single dose of 25 μg of a genistein-mab conjugate provided complete protection from a lethal challenge with the B cell leukemia. CD 19 is a B lineage specific surface molecule; the antibody conjugate was shown to induce apoptosis following internalization by receptor mediated endocytosis. T cell receptors are endocytosed following recognition of DR2/peptide complexes (Valitutti et al., 1995); it is therefore likely that multivalent DR2/peptide complexes will be taken up target T cells following binding to the T cell receptor.

Genistein will be conjugated in multivalent DR2/peptide complexes by photoaffinity crosslinking using a photosensitive 18.2 A long non-cleavable hetero-bifunctional crosslinking agent (Sulfo-SANPAH) as described by Uckun et al. (1995). The DR2-toxin conjugates will be tested using the human DR2 restricted T cell clones. T cells will be incubatedwith the DR2-toxin conjugates and the inductionof apoptosis will be assessed by agarose gel electrophoresis of genomic DNA. Nucleosomal fragmentation of DNA will be examined by ethidium bromide staining. DR2 molecules loaded with control peptides as well as control T cell clones will be used to demonstrate the specificity of apoptosis induction.

Apoptosis induction by DR2-toxin conjugates will be quantitated by flow cytometry following end labeling of fragmented DNA ends (TUNEL procedure). The free ends of nuclear DNA fragments will be labeled with dioxygenin-conjugated nucleotides, using the enzyme terminal deoxynucleotidyl transferase (TdT). Cells will be fixed and permeabilized by treatment with 70% EtOH. The 3′-OH ends of nuclear DNA fragments will be labeled with dioxygenin-dUTP, dioxygenin-dATP and TdT, followed by detection of labeled DNA ends with a fluorescein labeled anti-dioxygenin antibody (ApopTag, in situ apoptosis detection kit, Oncor). FACS analysis will be used to determine the fraction of cells that have undergone apoptosis. Cells grown for 12 hours at a low serum concentration (1% serum) will be used as a positive control. Specificity of apoptosis induction will be demonstrated by using control T cells clones and DR2 molecules loaded with control peptides.

5. T Cell Binding to Immobilized DR2/Peptide Complexes. Previous studies had demonstrated that recombinant, soluble DR2 molecules specifically bind peptides. To examine if recombinant DR2/peptide complexes are recognized by T cell receptors, T cell adhesion assays were performed using biotinylated DR2/peptide complexes that were captured on streptavidin coated microtiter plates. MBP(85-99) specific T cell clones and control T cell clones were labeled with BCEFC-AM, a fluorescent membrane probe, washed and incubated for 30 minutes at 37° C. with immobilized DR2/peptide complexes. Following washing, the fraction of bound T cells was determined in a fluorometer. Binding of MBP(85-99) specific, DR2 restricted T cells was only observed when DR2/MBP(85-99) complexes were used, but not when DR2 molecules were loaded with a control peptide. Also, a single amino acid substitution at a primary TCR contact residue in the peptide abolished T cell binding. Binding to DR2/MBP(85-99) complexes was not observed with control T cell clones.

Nucleic Acid Sequence encoding DR2-IgG fusion.

positions 1-15: 3′ end of secretory signal
positions 16-588: DRA*0101 extracellular domain
positions 589-609: linker
positions 610-729: Fos leucine zipper domain

positions 730-1437: IgG domain

ctcgagaaaa gagagatcaa agaagaacat gtgatcatcc aggccgagtt ctatctgaat cctgaccaat
caggcgagtt tatgtttgac tttgatggtg atgagatttt ccatgtggat atggcaaaga aggagacggt
ctggcggctt gaagaatttg gacgatttgc cagctttgag gctcaaggtg cattggccaa catagctgtg
gacaaagcca acttggaaat catgacaaag cgctccaact atactccgat caccaatgta cctccagagg
taactgtgct cacgaacagc cctgtggaac tgagagagcc caacgtcctc atctgtttca tagacaagtt
caccccacca gtggtcaatg tcacgtggct tcgaaatgga aaacctgtca ccacaggagt gtcagagaca
gtcttcctgc ccagggaaga ccaccttttc cgcaagttcc actatctccc cttcctgccc tcaactgagg
acgtttacga ctgcagggtg gagcactggg gcttggatga gcctcttctc aagcactggg agtttgatgc
tccaagccct ctcccagaga ctacagaggt cgacggaggt ggcggcggtt taactgatac actccaagcg
gagacagatc aacttgaaga cgagaagtct gcgttgcaga ccgagattgc caatctactg aaagagaagg
aaaaactgga gttcatcctg gccgcccatg cagcatctga gcccagaggg cccacaatca agccctgtcc
tccatgcaaa tgcccagcac ctaacctctt gggtggacca tccgtcttca tcttccctcc aaagatcaag
gatgtactca tgatctccct gagccccata gtcacatgtg tggtggtgga tgtgagcgag gatgacccag
atgtccagat cagctggttt gtgaacaacg tggaagtaca cacagctcag acacaaaccc atagagagga
ttacaacagt actctccggg tggtcagtgc Cctccccatc cagcaccagg actggatgag tggcaaggag
ttcaaatgca aggtcaacaa caaagacctc ccagcgccca tcgagagaac catctcaaaa cccaaagggt
cagtaagagc tccacaggta tatgtcttgc ctccaccaga agaagagatg actaagaaac aggtcactct
gacctgcatg gtcacagact tcatgcctga agacatttac gtggagtgga ccaacaacgg gaaaacagag
ctaaactaca agaacactga accagtcctg gactctgatg gttcttactt catgtacagc aagctgagag
tggaaaagaa gaactgggtg gaaagaaata gctactcctg ttcagtggtc cacgagggtc tgcacaatca
ccacacgact aagagcttct cccggactcc gggtaaatga gaattc

Nucleic Acid Sequence encoding DR2-IgM fusion.
positions 1-75: 3′ end of secretory signal
positions 76-648: DRA*0101 extracellular domain
positions 649-669: linker
positions 670-789: Fos leucine zipper domain

positions 790-1836: IgG domain

atggccataa gtggagtccc tgtgctagga tttttcatca tagctgtgct gatgagcgct caggaatcat
gggctatcaa agaagaacat gtgatcatcc aggccgagtt ctatctgaat cctgaccaat caggcgagtt
tatgtttgac tttgatggtg atgagatttt ccatgtggat atggcaaaga aggagacggt ctggcggctt
gaagaatttg gacgatttgc cagctttgag gctcaaggtg cattggccaa catagctgtg gacaaagcca
acttggaaat catgacaaag cgctccaact atactccgat caccaatgta cctccagagg taaetgtgct
cacgaacagc cctgtggaac tgagagagcc caacgtcctc atctgtttca tagacaagtt caccccacca
gtggtcaatg tcacgtggct tcgaaatgga aaacctgtca ccacaggagt gtcagagaca gtcttcctgc
ccagggaaga ccaccttttc cgcaagttcc actatctccc cttcctgccc tcaactgagg acgtttacga
ctgcagggtg gagcactggg gcttggatga gcctcttctc aagcactggg agtttgatgc tccaagccct
ctcccagaga ctacagaggt cgacggaggt ggcggcggtt taactgatac actccaagcg gagacagatc
aacttgaaga cgagaagtct gcgttgcaga ccgagattgc caatctactg aaagagaagg aaaaactqga
gttcatcctg gccgcccacg tcgcagaaat gaaccccaat gtaaatgtgt tcgtcccacc acgggatggc
ttctctggcc ctgcaccacg caagtctaaa ctcatctgcg aggccacgaa cttcactcca aaaccgatca
cagtatcctg gctaaaggat gggaagctcg tggaatctgg cttcaccaca gatccggtga ccatcgagaa
caaaggatcc acaccccaaa cctacaaggt cataagcaca cttaccatct ctgaaatcga ctggctgaac
ctgaatgtgt acacctgccg tgtggatcac aggggtctca ccttcttgaa gaacgtgtcc tccacatgtg
ctgccagtcc ctccacagat atccttaatt ttactattcc tccttccttt gccgacatct tccttagcaa
gtccgctaac ctgacctgtc tggtctcaaa cctggcaacc tatgaaaccc tgagtatctc ctgqgcttct
caaagtggtg aaccactgga aaccaaaatt aaaatcatgg aaagccatcc caatggcacc ttcagtgcta
agggtgtggc tagtgtttgt gtggaagact ggaataacag gaaggaattt gtgtgtactg tgactcacag
ggatctgcct tcaccacaga agaaattcat ctcaaaaccc aatgaggtgc acaaacatcc acctgctgtg
tacctgctgc caccagctcg tgaacaactg aacctgaggg agtcagccac agtcacctgc ctggtgaagg
gcttctctcc tgcagacatc tctgtgcaat ggaagcagag gggccagctc ttaccccagg agaagtatgt
gaccagtgcc ccgatgccag agcctggggc cccaggcttc tactttaccc acagcatcct gactgtgaca
gaggaggaat ggaactccgg agagacctat acctgtgttg taggccacga ggccctgcca cacctggtga
ccgagaggac cgtggacaag tccactggta aacccacact gtacaatgtc tccctgatca tgtctgacac
aggcggcacc tgctattgaa gatctgtcga c

### REFERENCES

Acha-Orbea et al. (1988) Cell 54:263-273.
Angel et al. (1988) Nature 332:166-171.
Arano et al. (1991) Bioconj. Chem. 2:71-76.
Arulanandam et al. (1993) J. Exp. Med. 177:1439-1450.
Avva and Cresswell (1994) Immunity 1:763-774.
Bjorkman et al. (1987) Nature 329:512-518.
Bodmer et al. (1995) Tissue Antigens 46:1-18.
Brake (1990) Meth. Enzymol. 185:408-421.
Brown et al. (1993) Nature 364:33-39.
Bunch et al. (1988) Nucl. Acid Res. 16:1043-1061.
Chang et al. (1994) Proc. Natl. Acad. Sci. (USA) 91:11408-11412.
Chicz et al. (1992) Nature 358:764-768.
Chicz et al. (1993) J. Exp. Med. 178:27-47.
Chou et al. (1989) J. Neurosci. Res. 23:207-216.
Corr et al. (1994) Science 265:946-949.
Cosson and Bonifacino (1992) Science 258:659-662.
Cregg et al. (1987) Bio/Technology 5:479-485.
Crothers and Metzger (1972) Immunochemistry 9:341-357.
Davies et al. (1994) Nature 371:130-136.
Dower et al. (1984) J. Immunol. 132:751-758.
Ebers (1996) IBC's Fourth Annual International Conference: Advances in the Understanding and Treatment of Multiple Sclerosis. Meeting Abstract, San Francisco June 1996.
Ferré-D'Amaré et al. (1993) Nature 363:38-45.
Gorga et al. (1987) J. Biol. Chem. 262:16087-16094.
Gregiore et al. (1991) Proc. Natl. Acad. Sci. (USA) 88:8077-8081.
Hammer et al. (1994) J. Exp. Med. 180:2353-2358.
Hammer et al. (1995) J. Exp. Med. 181:1847-1855.
Hauser (1996) IBC's Fourth Annual International Conference: Advances in the Understanding and Treatment of Multiple Sclerosis. Meeting Abstract, San Francisco June 1996.
Heldin (1995) Cell 80:213-223.
Hilbert et al. (1994) J. Neuroimmunol. 50:95-100.
Holz et al. (1996) J. Neurosci. 16:467-477.
Hornick and Karush (1972) Immunochemistrv 9:325-340.
Hu et al. (1990) Science 250:1400-1403.
Jardetzky et al. (1996) Proc. Natl. Acad. Sci. (USA) 93:734-738.
Kabat et al. (1979) “Sequences of Immunoglobulin Chains: Tabulation and Analysis of Amino Acid Sequences of Precursors, V-Regions, C-Regions, J-Chain and β₂-Microglobulins,” NIH Publication No. 80-2008, U.S. Dept. of Health Education and Welfare.
Kuby (1994) Immunology, 2nd Edition, W.H. Freeman and Company, New York.
Leonard et al. (1984) Nature 311:626-631.
Manolios et al. (1990) Science 249:274-277.
Marrosu et al. (1988) Neurology 38:1749-1753.
Marsh and Bodmer (1995) Tissue Antigens 45:258-280.
Matsui et al. (1991) Science 254:1788-1791.
Matsuuchi et al. (1986) Proc. Natl. Acad. Sci. (USA) 83:456-460.
Oas and Endow (1994) TIBS 19:51-54.
O'Shea et al. (1989) Science 245:646-648.
O'Shea et al. (1991) Science 254:539-544.
Pack and Pluckthun (1992) Biochemistry 31:1579-1584.
Pette et al. (1990) Proc. Natl. Acad. Sci. (USA) 87:7968-7972.
Potter et al. (1993) Int. Rev. Immunol. 10: 103-112.
Roberts et al. (1996) J. Biomed. Mat. Res. 30:53-56.
Sato et al. (1989) Biochem. Biophys. Res. Comm. 163:1473-1480.
Schatz (1993) Bio/Technolog 11: 1138-1143.
Sharma et al. (1991) Proc. Natl. Acad. Sci. (USA) 88:11465-11469.
Spielman and Nathenson (1982) Epidemiol. Rev. 4:45-65.
Steinman (1996) Cell 85:299-302.
Stem and Wiley (1992) Cell 68:465-477.
Stern et al. (1994) Nature 368:215-221.
Strorninger and Wiley (1995) JAMA 274:1074-1076.
Suda et al. (1993) Cell 75:1169-1178.
Sykulev et al. (1994) Proc. Natl. Acad. Sci. (USA) 91:11487-11491.
Todd et al. (1988) Science 240:1003-1009.
Uckun et al. (1995) Science 267:886-891.
Valitutti et al. (1995) Nature 375:148-151.
Valli et al. (1993) J. Clin. Invest. 91:616-628.
van Straaten et al. (1983) Proc. Natl. Acad. Sci. (USA) 80:3183-3187.
Viskochil et al. (1991) Mol. Cell. Biol. 11:906-912.
Weber et al. (1992) Nature 356:793-796.
Wettstein et al. (1991) J. Exp. Med. 174:219-228.
Wood et al. (1990) J. Immunol. 145:3011-3016.
Wucherpfennig and Strominger (1995a) Cell 80:695-705.
Wucherpfennig and Strominger (1995b) J. Bxp. Med. 181:1597-1601.
Wucherpfennig et al. (1991) Immunol. Today 12:277-282.
Wucherpfennig et al. (1994a) J. Bxp. Med. 179:279-290.
Wucherpfennig et al. (1994b) J. lImunol. 150:5581-5592.
Wucherpfennig et al. (1995) Proc. Natl. Acad. Sci. (USA) 92:8896-8900.

1. A Class II Major Histocompatibility Complex fulsion protein comprising a fusion of, toward the N-terminus, at least an MHC Class II binding domain of an MHC Class II α chain and, towards the C-terminus, a dimerization domain.

2-102. (Canceled)

103. A method for detecting T-cells in a host, comprising:
(a) contacting a population of T-cells from said host with a multimeric Major Histocompatibility Complex (MHC) binding domain conjugate, wherein said conjugate comprises:
(1) a carrier; and
(2) about 5 to 500 MHC binding domains conjugated to said carrier, wherein said MHC binding domains are bound to MHC-binding peptides; and,
(b) detecting the presence or absence of T-cells bound to said multimeric MHC binding domain conjugate, wherein said T-cells are specific for said MHC-binding peptides.

104. The method of claim 103, further comprising isolating T-cells bound to said conjugate.

105. The method of claim 104, further comprising administering to a subject the isolated T-cells to confer said subject with adoptive immunity against-said MHC-binding peptides.

106. The method of claim 103, wherein said carrier is labeled with a first fluorescent label.

107. The method of claim 103, wherein said MHC-binding peptides are covalently bound to said MHC binding domains.

108. The method of claim 103, wherein said MHC-binding peptides are non-covalently bound to said MHC binding domains.

109. The method of claim 103, wherein said MHC-binding peptides are labeled with a second fluorescent label.

110. The method of claim 103, wherein about 10 to about 200 MHC binding domains are conjugated to said carrier.

111. The method of claim 103, wherein about 20 to about 100 MHC binding domains are conjugated to said carrier.

112. The method of claim 103, wherein said carrier defines a minimal surface and said MHC binding domains are present at an average density of about 4×10-3 to 20 MHC binding domains/nm² on said surface.

113. The method of claim 103, wherein said carrier defines a minimal surface and said MHC binding domains are present at an average density of about 4×10-2 to 20 MHC binding domains/nm² on said surface.

114. The method of claim 103, wherein said carrier defines a minimal surface and said MHC binding domains are present at an average density of about 0.4 to 20 MHC binding domains/nm² on said surface.

115. The method of claim 103, wherein said carrier has a maximum diameter of about 5 to about 1000 nm.

116. The method of claim 103, wherein said carrier has a maximum diameter of about 5 to 500 nm.

117. The method of claim 103, wherein said carrier has a maximum diameter of about 5 to about 100 nm.

118. The method of claim 103, wherein said carrier is about 100 kDa to about 10,000 kDa.

119. The method of claim 103, wherein said carrier is about 100 kDa to about 5,000 kDa.

120. The method of claim 103, wherein said carrier is about 100 kDa to about 1,000 kDa.

121. The method of claim 103, wherein said carrier is about 100 kDa to about 500 kDa.

122. The method of claim 103, wherein said conjugate is about 400 kDa to about 10,000 kDa.

123. The method of claim 103, wherein said conjugate is about 400 kDa to about 5,000 kDa.

124. The method of claim 103, wherein said conjugate is about 400 kDa to about 1,000 kDa.

125. The method of claim 103, wherein said conjugate is about 400 kDa to about 500 kDa.

126. The method of claim 103, wherein said carrier is a particulate.

127. The method of claim 103, wherein said carrier is biodegradable.

128. The method of claim 103, wherein said carrier is non-immunogenic.

129. The method of claim 103, wherein said carrier is a branched polymer.

130. The method of claim 103, wherein said carrier has a net negative charge.

131. The method of claim 103, wherein said carrier has no net charge.

132. The method of claim 103, wherein said carrier is covalently bound to said MHC binding domains.

133. The method of claim 103, wherein said carrier is non-covalently bound to said MHC binding domains.

134. The method of claim 103, wherein said carrier is a substantially spherical bead.

135. The method of claim 134, wherein said bead is porous.

136. The method of claim 134, wherein said bead comprises a material selected from the group consisting of glass, silica, polyesters of hydroxylcarboxylic acids, polyanhydrides of dicarboxylic acids, or copolymers of hydroxylcarboxylic acids, and dicarboxylic acids.

137. The method of claim 103, wherein said carrier comprises a branched polymer.

138. The method of claim 137, wherein said branched polymer is a dendrimer.

139. The method of claim 138, wherein said dendrimer defines a minimal surface, and wherein said surface has a net neutral or net negative charge.

140. The method of claim 139, wherein said dendrimer comprises a material selected from a group consisting of a polyamidoamine, a polyamidoalcohol, a polyalkleneimine, a polyaklylene, a polyether, a polythioether, a polyphosphonium, a polysiloxane, a polyamide, and a polyaryl polymer.

141. The method of claim 103, wherein said carrier is a liposome.

142. The method of claim 141, wherein said liposome comprises a material selected from the group consisting of phosphatidyl chlorine, phosphatidyl serine, phosphatidyl inositol, phosphatidyl glycerol, phosphatidyl ethanolamine, phosphatidic acid, dicetyl phosphate, monosialoganglioside, polyethylene glycol, stearyl amine, ovolecithin and cholesterol.

143. The method of claim 103, wherein each MHC binding domain comprises a heterodimer of at least the peptide binding domain of an MHC Class I (x chain and an MHC Class β chain.

144. The method of claim 103, wherein each MHC binding domain comprises a heterodimer of at least the peptide binding domain of an MHC Class II OL chain and an MHC Class II β chain.

145. The method of claim 103, wherein said MHC-binding peptides is associated with an autoimmune disease.

146. The method of claim 145, wherein said autoimmune disease is Pemphigus Vulgaris (PV), Rheumatoid Arthritis (RA), Multiple Sclerosis (MS), or Systemic Lupus Erythematosus (SLE).

147. The method of claim 103, wherein said MHC-binding peptides is selected from residues 85-99, 84-102, or 148-162 of human myelin basic protein; residues 78-93, 97-111, 190-204, 206-220, 251-265, 512-526, or 762-786 of human desmoglein 3 protein.

148. The method of claim 103, wherein said MHC-binding peptides bind said MHC binding domains under physiological conditions.

149. A method for detecting T-cells in a host, comprising:
(a) contacting a population of T-cells from said host with an MHC binding domain, wherein said MHC binding domain is bound to an MHC-binding peptide; and
(b) detecting the presence or absence of T-cells bound to a complex of said MHC binding domain and said MHC-binding peptide, wherein said T-cells are specific for said MHC-binding peptides.

150. The method of claim 149, wherein said MHC binding domain is monovalent or multivalent.

151. A method for detecting T-cells associated with an autoimmune disease in a host, comprising:
(a) contacting a population of T-cells from said host with a multimeric MHC binding domain conjugate, wherein said conjugate comprises:
(1) a carrier; and,
(2) about 5 to 500 MHC binding domains conjugated to said carrier, wherein said MHC binding domains are bound to MHC-binding peptides associated with said autoimmune disease; and,
(b) detecting the presence or absence of T-cell bound to said multimeric MHC binding domain conjugate;
wherein detection of binding is indicative of an autoimmune disease.

152. The method of claim 151, wherein said autoimmune disease is Pemphigus Vulgaris (PV), Rheumatoid Arthritis (RA), Multiple Sclerosis (MS), or Systemic Lupus Erythematosus (SLE).

153. A method for detecting T-cells associated with an autoimmune disease in a host, comprising:
(a) contacting a population of T-cells from said host with an MHC binding domain, wherein said MHC binding domain is bound to an MHC-binding peptide associated with said autoimmune disease; and,
(b) detecting the presence or absence of T-cells bound to a complex of said MHC binding domain and said MHC-binding peptide;
wherein detection of binding is indicative of an autoimmune disease.

154. The method of claim 153, wherein said autoimmune disease is PV, RA, MS, or SLE.

155. The method of claim 153, wherein said MHC binding domain is monovalent or multivalent.
